(19) United States
(12) Patent Application Publication
(10) Pub. No.: US 2024/0230691 A1
(43) Pub. Date: Jul. 11, 2024

(54) SYSTEMS AND METHODS FOR A REGULATORY-COMPLIANT AUTOMATED ASSAY

(71) Applicant: Regeneron Pharmaceuticals, Inc., Tarrytown, NY (US)

(72) Inventors: Yi Liu Chen, Albany, NY (US); Megan McCann, Mechanicville, NY (US); Owen Tang, Troy, NY (US); Cuiping Zhao, Watervliet, NY (US); Kristyn Greco, Cohoes, NY (US); Rong Wang, Latham, NY (US); Keith Lawyer, Rensselaer, NY (US)

(21) Appl. No.: 18/409,065

(22) Filed: Jan. 10, 2024

Related U.S. Application Data
(60) Provisional application No. 63/438,391, filed on Jan. 11, 2023.

(51) Int. Cl. G01N 35/00

(52) U.S. Cl. CPC G01N 35/00623; G01N 2035/00633

(57) ABSTRACT

The present invention pertains to methods and systems for automated regulatory-compliant assays. In particular, the present invention pertains, in part, to fully-automated methods and systems for conducting GMP-compliant cell-based bioassays.

### CROSS-REFERENCE TO RELATED APPLICATIONS

This application claims the benefit of U.S. Provisional Application No. 63/438,391, filed Jan. 11, 2023, which is incorporated by reference herein in its entirety.

### FIELD

The present invention relates to systems and methods for a regulatory-compliant automated assay.

### BACKGROUND

Discovery, development, production and quality control testing of biotherapeutic products require varied and time-consuming assays performed by trained operators. Automation can be used to improve the efficiency and reliability of a biotherapeutic assay, including semi-automated or fully-automated assays. Automating steps of an assay can also allow for an increase in throughput, allowing a laboratory to perform more assays in a given time period and reduce project timelines and costs.

The requirements for how an assay is best conducted, what types of samples are used, and what outcomes are measured can depend on whether the assay is, for example, at a research and development stage of therapeutic development, a quality control/assurance stage, or a manufacturing stage. Conducting an assay in a manner that is in compliance with regulations, for example Good Manufacturing Practices (GMP) guidelines, poses additional hurdles and challenges. To date, while automation has been used in laboratories at the research and development stage of therapeutic development, the challenges posed by meeting GMP-compliance mean that there does not yet exist a GMP-compliant fully-automated assay method or system.

Therefore, it will be appreciated that a need exists for regulatory-compliant fully-automated methods and systems for conducting biotherapeutic assays.

### SUMMARY

The present invention generally pertains to methods for automated assays. Laboratory assays such as cell-based bioassays typically require extensive hands-on time and frequently exhibit a high degree of variability due to, for example, the use of live cells, high dilution volumes and small pipetting volumes, which presents issues for both GMP testing in laboratories and manufacturing facilities as well as assay investigation. Various automated platforms have been developed to address assay variability and increase throughput at research and development scales. However, the hardware and software setup and validation investment required to implement these technologies have limited their incorporation into a GMP environment. Here, the development of automated GMP-compliant assays are described, decreasing the variability from manual steps.

This disclosure provides a method for conducting automated assays in a GMP-compliant manner. In some exemplary embodiments, the method comprises (a) a first component including a computer system that creates a protocol method, (b) a second component including an automated assay system, which contains hardware that can execute the protocol method created by the first component and is coupled to the first component, and (c) a third component coupled to the second component, the third component including a computer system that receives, creates and maintains a GMP-compliant dataset detailing the protocol executed by the automated assay system.

This disclosure further provides an automated GMP-compliant method for conducting an assay. In some exemplary embodiments, the method comprises developing a secure assay protocol for a GMP-compliant assay, storing the secured assay protocol to include any changes and record of associated usage data, wherein any change to the protocol is stored and tracked; communicating the secure assay protocol to a first secure computer system in accordance with the assay protocol; subjecting at least one sample to the secure assay protocol executed by the first secure computer system, wherein the first secure computer system causes an automated operation of the secure assay protocol on the sample; collecting data associated with the at least one sample subjected to the secure assay protocol; and generating a GMP-compliant dataset from the collected data, wherein the GMP-compliant dataset includes an audit trail of the dataset, identification of location data for the at least one sample throughout execution of the secure assay protocol, and a record of any changes to the secure assay protocol, software and/or equipment controlled by the first secure computer system.

In one aspect, the assay is a bioassay.

In one aspect, the protocol is optimized using data collected from at least one sample subjected to the protocol.

In one aspect, the protocol is protected by a password.

In one aspect, the protocol is subjected to quality control review between 1 and 31 times per month, between 1 and 10 times per month, between 1 and 7 times per week, 1 time per week, 2 times per week, or 3 times per week.

In one aspect, the first secure computer system executes the protocol using a scheduling software. In a specific aspect, the scheduling software is Cellario.

In one aspect, an automated operation of the protocol includes a robotic arm. In a specific aspect, the robotic arm is an ACell robotic arm.

In one aspect, an automated operation of the protocol includes at least one liquid handler and/or reagent dispenser. In a specific aspect, the at least one liquid handler and/or reagent dispenser is a Hamilton STARlet, and/or a Multidrop Combi Reagent Dispenser.

In one aspect, the at least one sample is selected from a group consisting of cell culture fluid, harvested cell culture fluid, filtrate, chromatography eluate, drug substance, and drug product.

In one aspect, the at least one sample includes at least one therapeutic protein, wherein said protein is selected from a group consisting of an antibody, a monoclonal antibody, a bispecific antibody, a fusion protein, an antibody-drug conjugate, a receptor, and an antibody fragment. In a specific aspect, the at least one therapeutic protein is imdevimab or casirivimab. In another specific aspect, the at least one therapeutic protein is dupilumab.

In one aspect, collecting data comprises subjecting the at least one sample to at least one measurement. In a specific aspect, the at least one measurement is selected from a group consisting of spectrophotometry, absorbance detection, ultraviolet detection, fluorescence detection, luminescence detection, radioactivity detection, Raman spectroscopy, mass spectrometry, biolayer interferometry, and surface plasmon resonance.

In one aspect, the at least one sample is contained in a microplate.

In one aspect, collecting data includes using at least one data analysis software. In a specific aspect, the at least one data analysis software is SoftMax.

In one aspect, the dataset includes a unique identifier for the at least one sample. In another aspect, the dataset includes a unique identifier for a container containing said at least one sample.

In one aspect, the method further comprises generating a unique identifier for a container containing said at least one sample. In a specific aspect, the unique identifier is a barcode. In a more specific aspect, the barcode is generated using Sci-Print MP2+. In another specific aspect, the container is labeled with the barcode using Sci-Print MP2+. In an additional specific aspect, the method further comprises scanning the barcode at a critical step of subjecting the at least one sample to the secure assay protocol to generate location data for the at least one sample.

In one aspect, the dataset is stored in a comma-separated values file. In another aspect, the dataset can be stored in any format acceptable to the United States Food and Drug Administration, or comparable foreign equivalent.

In one aspect, the assay is a cell-based assay. In another aspect, the assay comprises isolation and/or purification of a therapeutic protein.

This disclosure also provides an automated system for conducting a GMP-compliant assay. In some embodiments, the system comprises a secure computer system, wherein the secure computer system stores a secure assay protocol for a GMP-compliant assay, and wherein the secure computer system is capable of collecting data associated with at least one sample subjected to the secure assay protocol and producing a GMP-compliant dataset; and at least one automated equipment capable of subjecting at least one sample to a secure assay protocol, wherein the secure computer system causes automated operation of the at least one automated equipment, wherein the GMP-compliant dataset includes an audit trail of the dataset, identification of location data for the at least one sample throughout execution of the secure assay protocol, and a record of any changes to the secure assay protocol, software and/or equipment controlled by the secure computer system.

In one aspect, the assay is a bioassay.

In one aspect, the protocol is created using Cellario.

In one aspect, the protocol is optimized using data collected from at least one sample subjected to the protocol.

In one aspect, the protocol is protected by a password.

In one aspect, the protocol is subjected to quality control review between 1 and 31 times per month, between 1 and 10 times per month, between 1 and 7 times per week, 1 time per week, 2 times per week, or 3 times per week.

In one aspect, the secure computer system causes automated operation of the at least one automated equipment using a scheduling software. In a specific aspect, the scheduling software is Cellario.

In one aspect, the at least one automated equipment includes a robotic arm. In a specific aspect, the robotic arm is an ACell robotic arm.

In one aspect, the at least one automated equipment includes at least one liquid handler and/or reagent dispenser. In a specific aspect, the at least one liquid handler and/or reagent dispenser is a Hamilton STARlet, a Multidrop Combi Reagent Dispenser, and/or a TEMPEST Liquid Handler.

In one aspect, the at least one sample is selected from a group consisting of cell culture fluid, harvested cell culture fluid, filtrate, chromatography eluate, drug substance, and drug product.

In one aspect, the at least one sample includes at least one therapeutic protein, wherein said protein is selected from a group consisting of an antibody, a monoclonal antibody, a bispecific antibody, a fusion protein, an antibody-drug conjugate, a receptor, and an antibody fragment. In a specific aspect, the at least one therapeutic protein is imdevimab or casirivimab. In another specific aspect, the at least one therapeutic protein is dupilumab.

In one aspect, collecting data comprises subjecting the at least one sample to at least one measurement. In a specific aspect, the at least one measurement is selected from a group consisting of spectrophotometry, ultraviolet detection, fluorescence detection, absorbance detection, luminescence detection, radioactivity detection, Raman spectroscopy, mass spectrometry, biolayer interferometry, and surface plasmon resonance.

In one aspect, the at least one sample is contained in a microplate.

In one aspect, collecting data includes using at least one data analysis software. In a specific aspect, the at least one data analysis software is SoftMax.

In one aspect, the dataset includes a unique identifier for said at least one sample.

In one aspect, the dataset includes a unique identifier for a container containing said at least one sample. In a specific aspect, the unique identifier is a barcode. In a more specific aspect, the barcode is generated using Sci-Print MP2. In another specific aspect, the container is labeled with the barcode using Sci-Print MP2. In an additional specific aspect, the automated operation comprises scanning the barcode at each step of subjecting the at least one sample to the secure assay protocol to generate location data for the at least one sample.

In one aspect, the dataset is stored in a comma-separated values file.

In one aspect, the assay is a cell-based assay. In another aspect, the assay comprises isolation and/or purification of a therapeutic protein.

### BRIEF DESCRIPTION OF THE DRAWINGS

FIG. 1 shows a high-level diagram of three key components of the present disclosure, according to an exemplary embodiment.

FIG. 2 shows a high-level flowchart of a method to create a GMP-compliant dataset, according to an exemplary embodiment.

FIG. 3 shows a decision tree of when a bioassay protocol will be updated and communicated to a secure computer system, according to an exemplary embodiment.

FIG. 4 shows a workflow of tracking a sample through an automated assay to produce a GMP-compliant dataset, according to an exemplary embodiment.

FIG. 5 shows a workflow of recording changes to equipment used in an automated assay to produce a GMP-compliant dataset, according to an exemplary embodiment.

FIG. 6 shows a flow chart of assay automation coupled with data automation, according to an exemplary embodiment.

FIG. 7 shows a setup of a fully automated assay system, according to an exemplary embodiment.

FIG. 8 shows a workflow of a fully automated assay system, according to an exemplary embodiment.

FIG. 9 shows a workflow of a semi-automated assay system, according to an exemplary embodiment.

FIG. 10 shows a plate format for an automated assay, according to an exemplary embodiment.

FIG. 11A shows a programmed method on liquid handler for an automated assay, according to an exemplary embodiment.

FIG. 11B shows code development on liquid handler for an automated assay, according to an exemplary embodiment.

FIG. 11C shows a 3D assay deck layout on liquid handler for an automated assay, according to an exemplary embodiment.

FIG. 11D shows a 2D assay deck layout on liquid handler for an automated assay, according to an exemplary embodiment.

FIG. 12 shows time savings between an automated assay and a manual assay, according to an exemplary embodiment.

FIG. 13A shows reference standard unconstrained R² for an automated anti-SARS-CoV-2 assay compared to a manual assay using imdevimab, according to an exemplary embodiment.

FIG. 13B shows reference standard unconstrained R² for an automated anti-SARS-CoV-2 assay compared to a manual assay using casirivimab, according to an exemplary embodiment.

FIG. 13C shows reference standard Max/Min Ratio for an automated anti-SARS-CoV-2 assay compared to a manual assay using imdevimab, according to an exemplary embodiment.

FIG. 13D shows reference standard Max/Min Ratio for an automated anti-SARS-CoV-2 assay compared to a manual assay using casirivimab, according to an exemplary embodiment.

FIG. 14A shows reportable potency of an automated anti-SARS-COV-2 assay compared to a manual assay using imdevimab, according to an exemplary embodiment.

FIG. 14B shows reportable potency of an automated anti-SARS-COV-2 assay compared to a manual assay using casirivimab, according to an exemplary embodiment.

FIG. 15A shows an assessment of position bias for an automated anti-SARS-COV-2 assay using imdevimab, according to an exemplary embodiment.

FIG. 15B shows an assessment of position bias for an automated anti-SARS-COV-2 assay using casirivimab, according to an exemplary embodiment.

FIG. 16A shows variability of an automated anti-SARS-COV-2 assay compared to a manual assay using casirivimab, according to an exemplary embodiment.

FIG. 16B shows variability of an automated anti-SARS-COV-2 assay compared to a manual assay using imdevimab, according to an exemplary embodiment.

FIG. 17A shows overall linearity of relative potency values obtained with an automated anti-SARS-COV-2 assay using imdevimab, according to an exemplary embodiment.

FIG. 17B shows overall linearity of relative potency values obtained with an automated anti-SARS-COV-2 assay using casirivimab, according to an exemplary embodiment.

FIG. 18A shows a variance component analysis of an automated anti-SARS-COV-2 assay using imdevimab, according to an exemplary embodiment.

FIG. 18B shows a variance component analysis of an automated anti-SARS-COV-2 assay using casirivimab, according to an exemplary embodiment.

FIG. 19A shows a summary of side-by-side tests and linearity tests of an automated anti-SARS-COV-2 assay using imdevimab, according to an exemplary embodiment.

FIG. 19B shows a summary of side-by-side tests and linearity tests of an automated anti-SARS-COV-2 assay using casirivimab, according to an exemplary embodiment.

### DETAILED DESCRIPTION

The processes of discovery, development, production and quality control testing of biotherapeutic products require a varied and complex array of time-consuming assays to, for example, identify, characterize and test a biotherapeutic product. Each assay may include the use of various reagents and one or more pieces of equipment, the complexity and sensitivity of which can provide challenges both for manual and automated operation of an assay.

An exemplary class of assays includes bioassays, which in the general sense used herein involve assessing a molecule or substance by biological methods, including through the use of living cells. Bioassays provide important information concerning the safety and potency of biological or pharmaceutical products. This is necessary in the field of drug development to evaluate the consistency among batches and stability of drug productions. Bioassays are generally used in research, clinical, environmental, and industrial settings to detect or quantify a presence or amount of certain gene sequences, antigens, diseases, proteins, peptides, and/or pathogens. Bioassays may be used to identify organisms including parasites, fungi, bacteria, and viruses present in a host organism or a sample. For example, bioassays can provide a measure of quantification which may be used to calculate the extent of infection or disease and to monitor the state of a disease over time. Accordingly, bioassays may provide a measure of quantification used to characterize an effect or quality of a therapeutic product.

Assays may feature manual steps performed by an analyst, automated steps performed by a machine, and combinations thereof. Automation may improve the overall efficiency and reliability of a system or method, reducing the amount of analyst time and effort required. For example, automation of a bioassay could allow for liquid handling to be more consistent and eliminate operator-to-operator error. Automation of assay workflows may also allow for an increase in throughput, allowing for a laboratory to perform more assays in a given time period, thus reducing project timelines and costs. However, the design of an automated system may present challenges based on the complexity and sensitivity of tasks to be performed, and based on the need to integrate the functions of a variety of different equipment.

Automation in a laboratory setting often includes the use of computer systems, robotic systems, and/or components. Robotic systems and components have been implemented in various related industries. For example, robotic systems and components are commonly used in the manufacturing of consumer goods such as automotive, electronics, pharmaceuticals, and biotechnology products. Robotic systems and components are often employed in biotechnology, medical, and laboratory settings to automate specific steps in an assay or bioassay process.

While automation is used in laboratory settings, for example at a research and development stage of pharmaceutical development, there does not currently exist an automated system capable of performing a bioassay in a manner that is GMP-compliant.

Cell-based bioassays typically require extensive hands-on time and frequently exhibit a high degree of variability due to the use of live cells, high dilution volumes and small pipetting volumes, which creates obstacles for both GMP routine testing in a QC lab and assay investigation. Various automated platforms have been developed to address bioassay variability and increase throughput in the research and development (R&D) environment. However, the hardware qualification and software validation investment required to implement these technologies have limited their incorporation into a GMP environment. Conforming to GMP standards requires additional layers of oversight regarding protocol, system, and sample handling and modifications, and software capable of integrating a complex physical process with a regulatory-compliant data collection and organization process.

Disclosed herein is the development of methods and systems for regulatory-compliant fully-automated assays. In some exemplary embodiments, an assay of the present invention is a GMP-compliant automated bioassay using an Integrated Laboratory Automated System (ILAS) platform, in which a majority of steps are automated, which decreases the variabilities from manual steps. Data from dilutional linearity studies and side-by-side manual vs. fully-automated assay comparison studies are presented. The results indicate that, in some exemplary embodiments, the fully-automated methods exhibit reliable dilutional linearity and comparable performance with a manual method, while achieving an 85% reduction in analyst hands-on time and a 95% reduction in analyst pipetting. The invalid rate was also compared between a fully-automated method developmental study and a manual method validation study. The results demonstrated that the invalid rate of a fully-automated method of the present invention significantly decreased compared to that of a manual assay. Taken together, the results demonstrate that the fully-automated assays of the present invention represent a viable replacement for manual assays with regards to assay performance in a GMP environment. Additionally, the automated methods and systems of the present invention provide better reproducibility and decreased human error, which makes automation a reliable tool for assay and bioassay investigation as well.

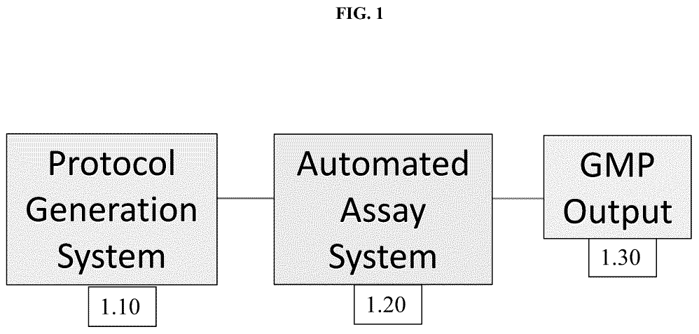

Aspects of the present disclosure include systems and methods for discovery, production, isolation and/or analysis of pharmaceutical products. According to certain embodiments, provided are fully automated assay systems. As shown in FIG. 1, the fully automated assay systems include a first computer system 1.10. The fully automated assay systems further include a second module 1.20 comprising a secure computer system. The fully automated assay systems further comprise a third regulatory-compliant output system 1.30. The first and second modules can be connected via a secure computational connection, such that the secure assay protocol can be passed from the protocol generation module 1.10 to the secure computer system 1.20, allowing the secure computer system 1.20 to execute the assay in accordance with the protocol. The second and third modules 1.20 and 1.30 can be similarly connected via a secure computation connection, such that the secure computer system 1.20 can pass information related to any changes occurring in the second system to the third system, thus preserving a record of those changes. In another aspect, the protocol can be transmitted via secure connection.

Before the present systems and methods are described in greater detail, it is to be understood that the present disclosure is not limited to the particular embodiments described. It is also to be understood that the terminology used herein is for the purpose of describing the particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present invention, representative illustrative systems and methods are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present systems and methods. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limit of that range and any other stated or intervening value in that stated range including the endpoints of the stated range, is encompassed within the present systems and methods. The upper and lower limits of these smaller ranges may independently be included in the smaller ranges and are also encompassed within the systems and methods, subject to any specifically excluded limit in the stated range. Where the stated range includes one or both of the limits, ranges excluding either or both of those included limits are also included in the systems and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, an assay is an investigative procedure for qualitative assessment or quantitative measurement of the presence, amount, or functional activity of at least one target analyte, often used in laboratories for research in medicine, pharmacology, environmental biology, or molecular biology. A target analyte may be a drug, a biochemical substance, a cell in an organism, or an organic sample, and the measured entity may be the analyte. A purpose of an assay is to measure a property of an analyte in discrete units, such as molarity, density, functional activity, or the degree of some effect in comparison to a standard. Additionally, an assay may yield qualitative results that may be interpreted by a skilled analyst.

Production of recombinant protein-based drug substances involves the development of several processes that adhere to the guidelines set forth by the United States Food and Drug Administration (FDA) referred to as current Good Manufacturing Practices (cGMP). cGMP-compliant, as used and defined herein, refers to a process that adheres to the FDA's cGMP guidelines. In order to sell a pharmaceutical composition or drug product in the U.S. and elsewhere, it is necessary to produce the pharmaceutical composition or drug product in cGMP compliance. Similarly, Good Manufacturing Practices (GMP) relates to GMP guidelines set forth by the FDA to ensure product quality and safety. Adherence to GMP guidelines requires, for example, ensuring that computerized systems are validated; ensuring that computer hardware and software are suitable to perform assigned tasks; providing controls to prevent unauthorized access or changes to data; providing a record of any data change made; batch production records including dates, times, equipment used, and results for each significant step in batch production; and laboratory control records including complete data derived from all tests conducted and a comparison to established acceptance criteria. In some exemplary embodiments, this disclosure provides automated methods and systems for conducting GMP-compliant assays.

As used herein, the term “data integrity” refers to the completeness, consistency, and accuracy of data collected by the system. As used herein, the term “GMP-compliant data integrity” refers to data kept attributable, legible, contemporaneously recorded, as an original or true copy, and accurate, as specified according to GMP standards.

As used herein, the term “metadata” refers to structured information that describes, explains, or otherwise facilitates the retrieval, use, or management of data.

As used herein, the term “audit trail” relates to a secure, computer-generated, time-stamped electronic record that allows for reconstruction of the course of events relating to the creation, modification, or deletion of an electronic record. In some exemplary embodiments, methods and systems of the present invention provide an automated audit trail in compliance with GMP guidelines. Generating an audit trail may include, for example, automatically providing a unique label for each container of each sample, automatically reading the unique label at each step of the assay, and subsequently automatically adding to a data file the time, date, location and other status of the sample container, for generation of a GMP-compliant dataset. Generating an audit trail may additionally include automatically storing data related to any changes to equipment or software involved in the assay, for generation of a GMP-compliant dataset. Entries added during a run of an assay may be referred to as a run audit trail, while entries added before, after, or between runs of an assay may be referred to as an outside run audit trail. The audit trail can be tracked in a LIMS system.

As used herein, the term “GMP-compliant backup” relates to a true copy of the original data generated from an assay that is maintained securely throughout the records retention period.

As used herein, the term “autosave” relates to the process of automatically saving or storing data into long-term storage at the time of performance.

As used herein, a “sample” can be obtained from any step of a bioprocess, such as cell culture fluid (CCF), harvested cell culture fluid (HCCF), any step in the downstream processing, drug substance (DS), or a drug product (DP) comprising a final formulated product.

As used herein, the term “scheduling software” refers to a software for scheduling jobs for software and equipment used in an assay. Scheduling software may receive, store, and/or provide instructions for performing an assay, for example an assay protocol. Scheduling software may provide instructions to integrate the jobs of, for example, robotic arms, liquid handlers, reagent dispensers, plate labelers, incubators, shakers, centrifuges, peelers, sealers, washers, heaters, plate lid handlers, barcode scanners, pipets, and/or detectors. Scheduling software may further integrate software for performing an assay, for example, a protocol development or storage software, software for operating equipment, and software for data collection and data analysis. In some exemplary embodiments, assays of the present invention use Cellario scheduling software.

### Systems

Aspects of the present disclosure include sample analysis systems. The analysis systems may be adapted to perform a variety of analyses of interest, including hematology analysis, slide preparation and cell morphology analysis, erythrocyte sedimentation rate (ESR) analysis, blood coagulation analysis, real-time nucleic acid amplification analysis, immunoassay analysis, clinical chemistry analysis, and combinations thereof. In certain aspects, the analysis systems are automated, meaning that the system is capable of performing sample analysis and any necessary sample preparation steps without user intervention.

According to certain embodiments, the system of the present disclosure may function by developing a secure assay protocol, which is then saved in permanent storage. The secure assay protocol is then employed by a sample testing system, which may be comprised of various pieces of equipment that execute the steps of the assay in accordance with the protocol. At each step of the process, the protocol saved in permanent storage may be updated via autosave, or manually saved by a user, with an audit trail of the execution of the protocol, and any deviations from the original protocol are cataloged in the permanent storage.

According to certain embodiments, the system of the present disclosure will follow a decision tree to update the assay protocol saved in permanent storage. After the initial assay protocol is stored, if any changes are encountered to the protocol during the execution of the assay, the system will create a record of the change and autosave it to the dataset stored in permanent storage.

According to certain embodiments, the secure computer system will function by initially cataloging samples via barcode. During the execution of the assay, the system will read the barcode at critical steps of a protocol, and store the location along with a date and time stamp to the dataset stored in permanent storage. This process will iterate for critical steps until the assay protocol is completed, after which a finalized GMP compliant dataset will be exported.

According to certain embodiments, during the execution of the assay, the secure computer system will create an initial save of the assay protocol including any necessary equipment specified by the assay protocol. During the protocol, the secure computer system can determine if a change has been made to the equipment in the system. If a change has been made, a record of the change will be created and autosaved to the dataset stored in permanent storage. This process will iterate for critical steps until the assay protocol is completed, after which a finalized GMP compliant dataset will be exported.

According to certain embodiments, the secure computer system is an automated system for the completion of an assay.

According to certain embodiments, the automated assay system is designed to perform automated bioassays. The system may be scalable and process whole sample specimens to produce a result containing information on relevant parameters.

According to certain embodiments, a system can function as a separate automated assay system, or function as part of an integrated system (e.g. configured in a work cell) with one or more other such automated assay systems.

An automated, GMP-compliant system according to one embodiment is shown in FIG. 1. In this configuration, a first module comprising a secure computer system creates a protocol to be executed as an assay. The protocol is then transmitted via secure connection to the second module, which comprises an automated assay system containing the necessary hardware to execute the assay protocol. During execution of the protocol, the second module transmits information of its actions to a third module, comprising a computer system which receives the information and catalogs it to create a GMP-compliant dataset.

According to certain embodiments, an assay protocol can be created via input from a user in the first secure computer system. Once the protocol is created, it is transmitted to the automated assay system, and to the second secure computer system which stores it for GMP compliance.

According to certain embodiments, once the protocol has been created and the initial protocol is stored, the automated assay is executed. The subject samples are input into the system and run through the automated assay system in the second module. During each step of the process, the secure assay system module communicates with the third module to catalog any deviations or changes made in the protocol from the original input protocol.

According to certain embodiments, at the termination of the process in the automated assay system, a dataset containing the initial assay protocol and the record of changes developed throughout the operation of the automated assay system is created.

In some exemplary embodiments, the first secure computer system can comprise a personal computer (“P.C.”) through which an operator can design a protocol for the execution of a assay.

In some exemplary embodiments, the transmission of an assay protocol can include the execution of pre-existing software code that, based on user input, passes a series of pre-existing protocols to an automated assay system.

In some exemplary embodiments, the automated assay system contains connected, either directly or indirectly, laboratory equipment that can be used in succession to execute the assay protocol.

In some exemplary embodiments, the automated assay system contains at least one robotic arm to assist in the transferring of samples and plates between laboratory equipment.

In some exemplary embodiments, the automated assay system includes automated pipets, which automate the liquid handling of reagents and reactants in accordance with the assay protocol.

In some exemplary embodiments, the automated assay system includes a barcode scanner, to automate the validation of the correct reagents and reactants in the assay protocol.

In some exemplary embodiments, the automated assay system includes a PlateOrient device that is used to automate the correct alignment of sample plates in laboratory equipment used in accordance with the assay protocol.

In some exemplary embodiments, the automated assay system includes a LidValet to assist in the automated lidding and de-lidding of plates in accordance with the assay protocol.

In some exemplary embodiments, the automated assay system includes an automated liquid handling platform such as a Hamilton Microlab STARlet to ensure the liquid handling of the assay is in accordance with the assay protocol.

In some exemplary embodiments, the automated assay system includes a thermoshaker to mix the sample, reagents, and intermediates in accordance with the assay protocol.

In some exemplary embodiments, the automated assay system includes a centrifuge to mix the sample, reagents, and intermediates in accordance with the assay protocol.

In some exemplary embodiments, the automated assay system includes a thermal heat sealer for use in accordance with the assay protocol.

In some exemplary embodiments, the assay protocol is stored using GMP-compliant data integrity standards.

In some exemplary embodiments, the assay protocol is stored including all metadata generated by the system, saved contemporaneously with any data saved to the protocol.

In some exemplary embodiments, the audit trail and all data can be saved contemporaneously. The audit trail and all data can be saved, for example, in a database.

In some exemplary embodiments, the assay protocol is saved with a GMP-compliant backup.

In some exemplary embodiments, the assay protocol is autosaved when any changes are made to the protocol. In some exemplary embodiments, the assay protocol can be saved by a user when any changes are made to the protocol.

In one exemplary aspect, the disclosure provides a non-transitory computer readable medium storing instructions for causing a processor to perform a method for creating an assay protocol.

In one exemplary aspect, the disclosure provides a non-transitory computer readable medium storing instructions for causing a processor to perform a method for transmitting the assay protocol to a dataset in permanent storage.

In one exemplary aspect, the disclosure provides a non-transitory computer readable medium storing instructions for causing a processor to transmit the assay protocol to a secure assay automated system.

In one exemplary aspect, the disclosure provides a non-transitory computer readable medium storing instructions for causing a processor to transmit the assay protocol from the secure assay automated system to individual components in the secure assay automated system.

In one exemplary aspect, the disclosure provides a non-transitory computer readable medium storing instructions for causing a processor to determine if a change has been made in the secure assay automated system, and, if a change has occurred, updating the assay protocol with the change, and saving the updated assay protocol in a non-transitory computer readable medium.

### EXAMPLES

### Example 1. Design of Fully-Automated GMP-Compliant Assay Systems

This disclosure describes the development of methods and systems for automated GMP-compliant assays. A system of the present invention may be referred to as an integrated laboratory automation system (ILAS). An automated system of the present invention may offer a number of capabilities that enable GMP-compliant assay automation. One such capability is the generation of a run audit trail, comprising automatically recording the details of the assay as it is performed. Another capability is the generation of an outside run audit trail, comprising automatically recording any modifications made to the system outside of an assay run. A third capability is the tracking of samples as they move through the system, for example using a unique barcode for each sample plate. A fourth capability of the system of the present invention is integration of scheduling software and data recording and/or data analysis software to allow automatic saving of data generated by the assay. A fifth capability comprises integration of scheduling software and individual equipment software, for example liquid handling and/or dispensing software, to allow for the security of equipment parameters. A sixth capability comprises automatic security of an assay protocol, preventing unauthorized changes to the protocol and recording any authorized changes made. A seventh capability is the integration of a custom data file, for example a spreadsheet or a text file, corresponding to a sample, which may be automatically updated by the system and used for tracking a sample before, during, and after an automated assay.

In an exemplary embodiment, scheduling software used to control an automated assay system is Cellario (HighRes Biosolutions), equipment used to move plates through the system is an ACell robotic arm (HighRes Biosolutions), equipment used for the majority of automated liquid handling is a Hamilton STARlet (Hamilton), software used to control the assay protocol, data collection and data analysis is SoftMax (Molecular Devices), a reagent dispenser can be Multidrop Combi Reagent Dispenser (ThermoFisher), an additional liquid handler or reagent dispenser can be the TEMPEST Liquid Handler/Liquid Dispenser (FORMULATRIX), a label printer used to label a sample plate is the Sci-Print MP2+(Scinomix), and a data file used to track each sample plate is a comma-separated values (csv) file. Mechanical equipment used to carry out an assay protocol may also be referred to as hardware.

A general illustration of the methods and systems of the present invention is presented in FIG. 1. A secure protocol is used to provide instructions for conducting an automated assay. An automated assay produces GMP-compliant data as an output.

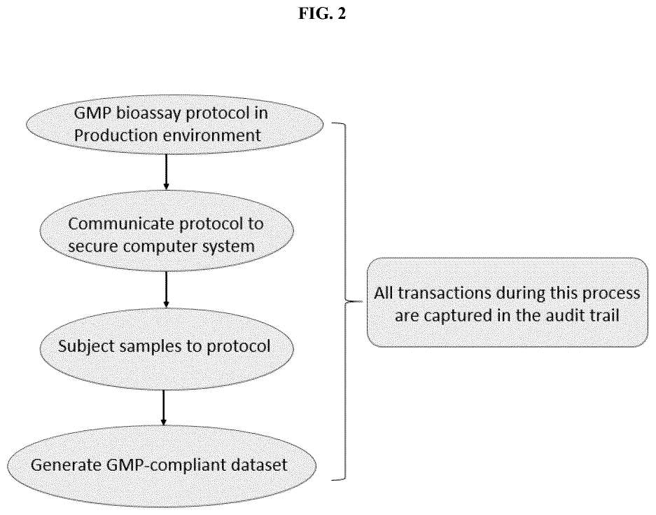

FIG. 2 further illustrates an embodiment of the present invention. The methods and systems of the present invention may including developing a secure assay protocol for a GMP-compliant assay; communicating the secure assay protocol to a secure computer system in accordance with the assay protocol; subjecting a sample to the secure assay protocol in an automated fashion using the secure computer system; and generating a GMP-compliant dataset using data collected from the sample subjected to the secure assay protocol. The dataset may further include an audit trail of the dataset, identification of location data for the sample throughout execution of the secure assay protocol, and a record of any changes to software, and/or equipment controlled by the computer system.

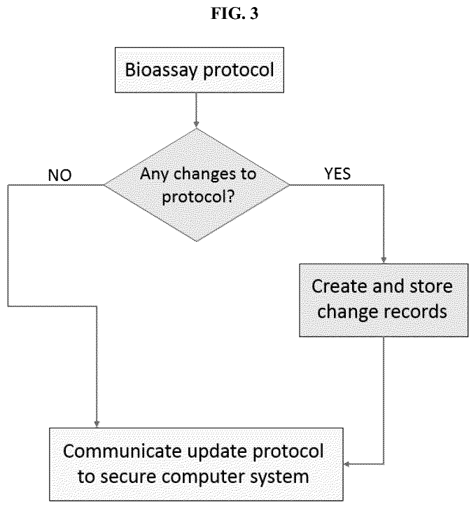

FIG. 3 further illustrates an embodiment of the present invention. Any changes to the assay protocol should result in creation and storage of records documenting the changes. A stored assay protocol may be communicated to a secure computer system in accordance with the assay protocol.

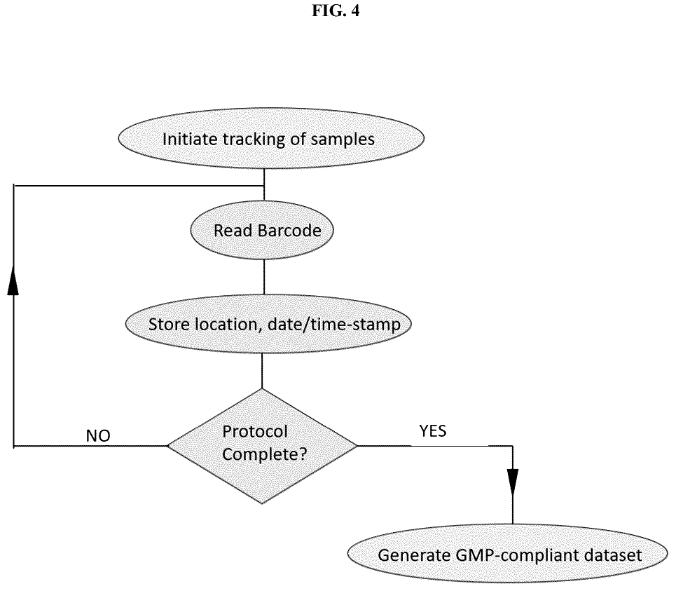

An additional embodiment of the present invention is illustrated in FIG. 4. Samples that will be subjected to the secure assay protocol are tracked, for example using a unique barcode affixed to a container containing the sample, as part of the generation of a run audit trail. Each time there is a change in the status or location of the sample, the automated system may read the barcode of the sample and store a record of the location, date and time of the sample. When the protocol is complete, these records may be incorporated into a GMP-compliant dataset pertaining to the sample.

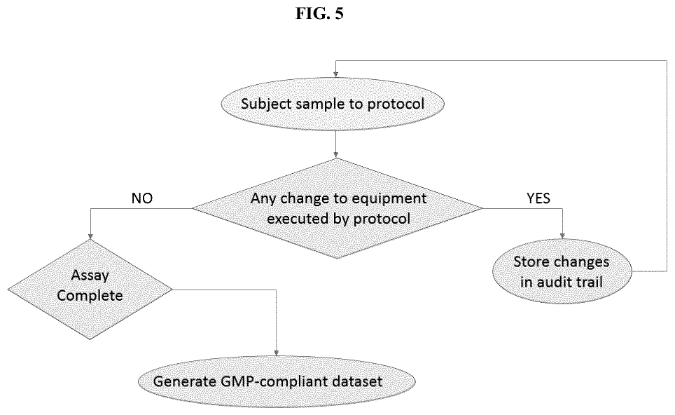

FIG. 5 illustrates another embodiment of the present invention. Before or after subjecting a sample to a secure assay protocol (also referred to as an assay run), any change to equipment involved in the assay may be recorded in an audit trail that will contribute to the GMP-compliant dataset upon completion of the protocol. This may also be referred to as an outside run audit trail.

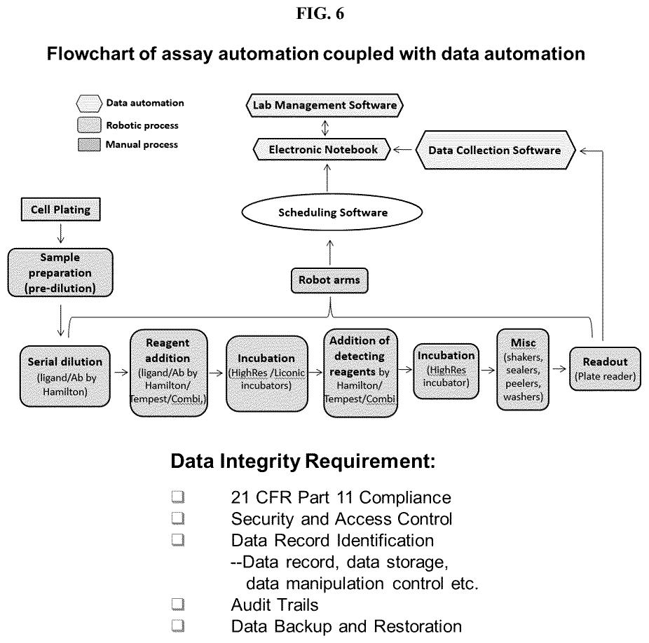

An illustration of automated features of methods and systems of the present invention is given in FIG. 6. Examples of automated sample processing steps are detailed. Examples of automated robotic processes include automated sample preparation; automated serial dilution using Hamilton STARlet; automated reagent addition using Hamilton STARlet, Tempest Liquid Handler and/or Multidrop Combi Reagent Dispenser; automated incubation using HighRes Biosolutions and/or Liconic incubators; automated addition of detecting reagents using Hamilton STARlet, Tempest Liquid Handler STARlet and/or Multidrop Combi Reagent Dispenser; use of additional automated equipment such as shakers, sealers, peelers and/or washers; automated plate reading; and automated robotic arms that are used, for example, to transport a sample between pieces of integrated automated equipment. Automated software steps may include automated data collection software; automated record-keeping in an electronic notebook; and automated lab management software. Requirements to be met to ensure data integrity include, for example, compliance with 21 C.F.R § 11; security and access control; data record identification; audit trails; and data backup and restoration.

### Example 2. Side-by-Side Comparison of Manual and Automated Assays for Anti-SARS-COV-2 Neutralization

In order to validate the automated GMP-compliant assay methods and systems of the present invention, an exemplary embodiment of an automated assay system of the present invention was developed for determining neutralization of SARS-COV-2 by two anti-SARS-CoV-2 antibodies, casirivimab (also known as REGN 10987) and imdevimab (also known as REGN 10933). Side-by-side comparison studies on an automated system (ILAS) compared to the manual assay for imdevimab and casirivimab were conducted.

The anti-SARS-COV-2 neutralization assay is an in vitro, cell-based assay that was developed to quantify the biological effects of anti-SARS-COV-2 antibodies imdevimab and casirivimab, in particular through neutralizing the SARS-COV-2 spike protein and preventing viral entry into cells via the ACE2 receptor. The assay uses Vero cells, which are an adherent cell line of epithelial kidney cells expressing a required component of SARS-COV-2 virus entry and infection of cells, the ACE2 receptor.

pVSV-Luc-SARS-COV-2-S pseudoparticles are used to represent the SARS-COV-2 virus. pVSV-Luc-SARS-COV-2-S pseudoparticles are vesicular stomatitis virus (VSV) virions, in which the VSV glycoprotein gene has been deleted and replaced with genes for the reporter proteins firefly luciferase (FLuc) and green fluorescent protein (GFP). These pVSV-Luc-G particles are pseudotyped with the SARS-COV-2 spike protein. pVSV-Luc-SARS-COV-2-S pseudoparticles are considered infectious, but are limited to a single round of infection mediated by the spike protein. Background infectivity is measured using pVSV-Luc pseudoparticles, which are pseudotyped without the spike protein.

In the anti-SARS-COV-2 assay, adherent Vero cells are plated and incubated overnight. On Day 2 of the assay, anti-SARS-COV-2 antibody is serially diluted and incubated with a constant amount of pVSV-Luc-SARS-COV-2-S pseudoparticles. The anti-SARS-COV-2 antibody/pseudoparticle complex is then added to the plated Vero cells and incubated overnight. During this incubation, the non-neutralized pseudoparticles will infect cells via the ACE2 receptor and activate a luciferase reporter in the Vero cells that results in luciferase expression. Following incubation, ONE-Glo is added to the plate wells to measure luciferase expression. Higher concentrations of anti-SARS-COV-2 products yield a lower luminescent signal due to more neutralization of the spike protein and subsequent lower binding to the ACE2 receptor. pVSV-Luc pseudoparticles, which lack a spike protein, are used as a negative control, and pVSV-Luc-SARS-COV-2-S pseudoparticles that are not incubated with anti-SARS-COV-2 antibody are used as a positive control. All pseudoparticles used herein may also be referred to as virus-like particles (VLP).

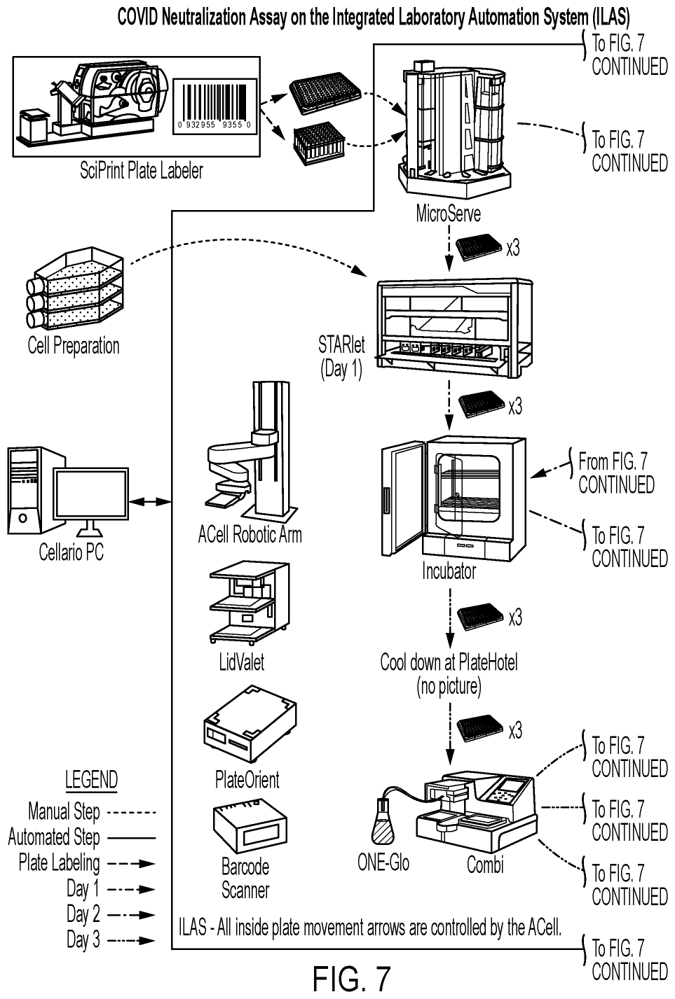

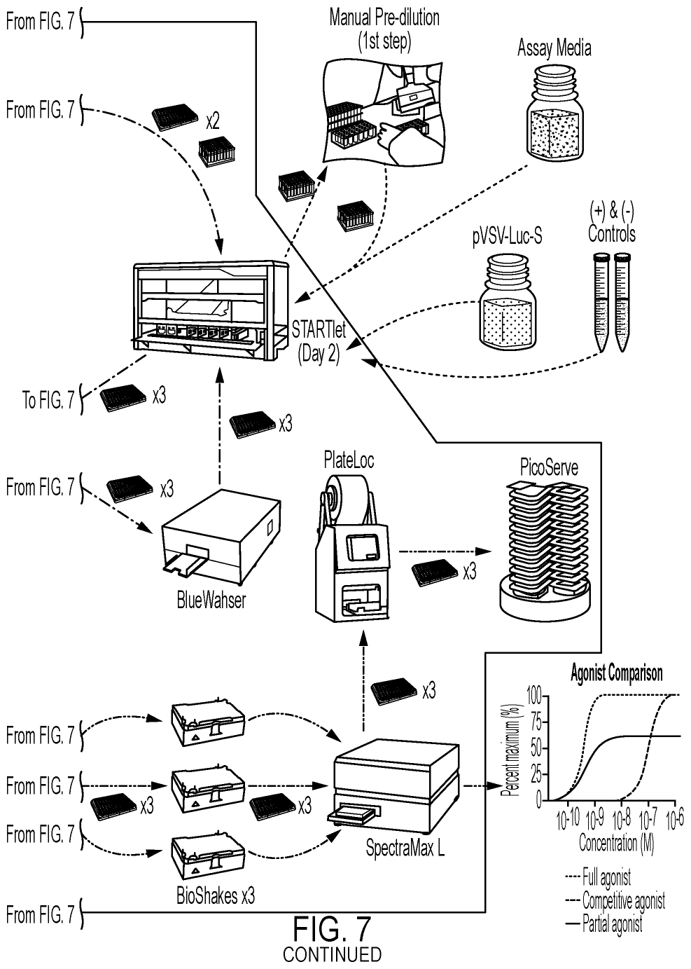

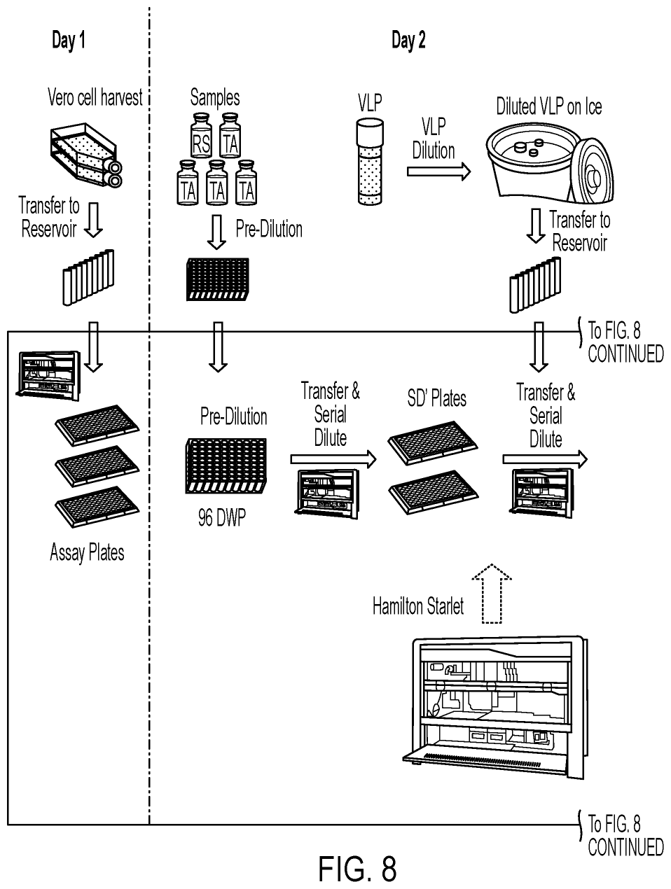

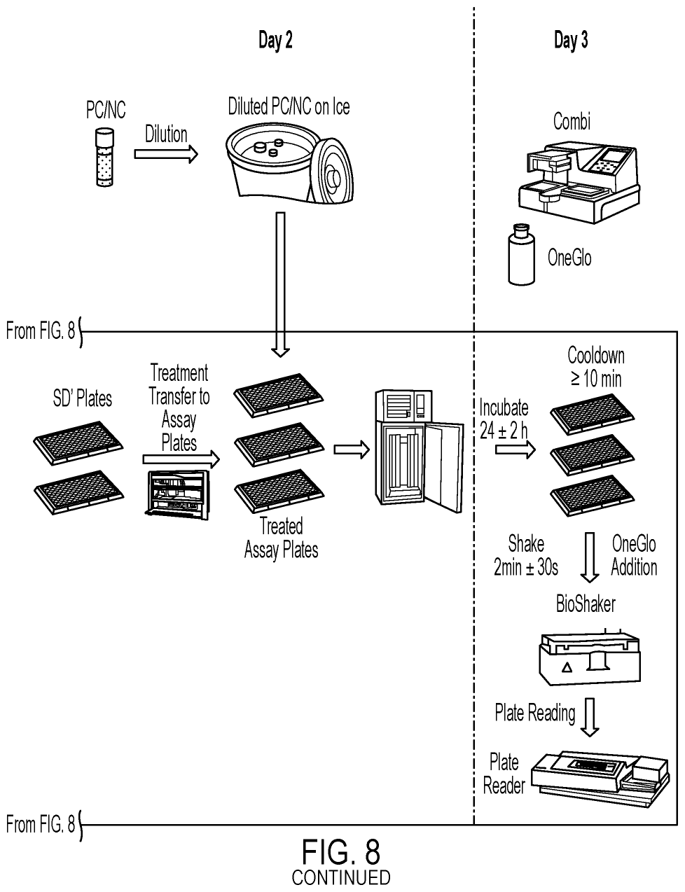

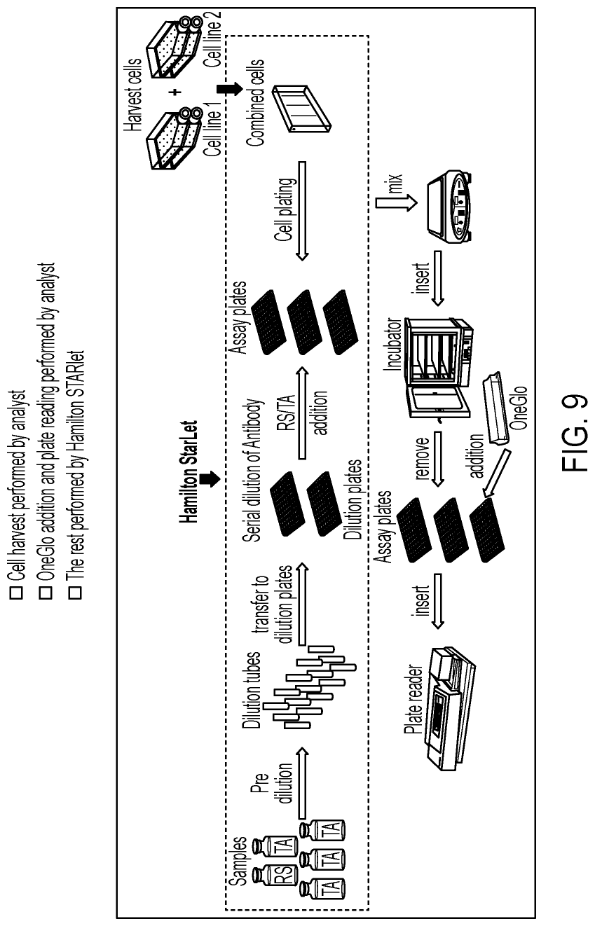

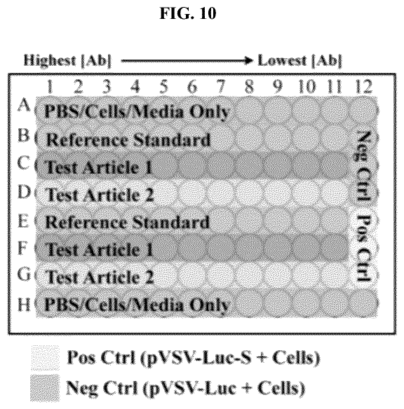

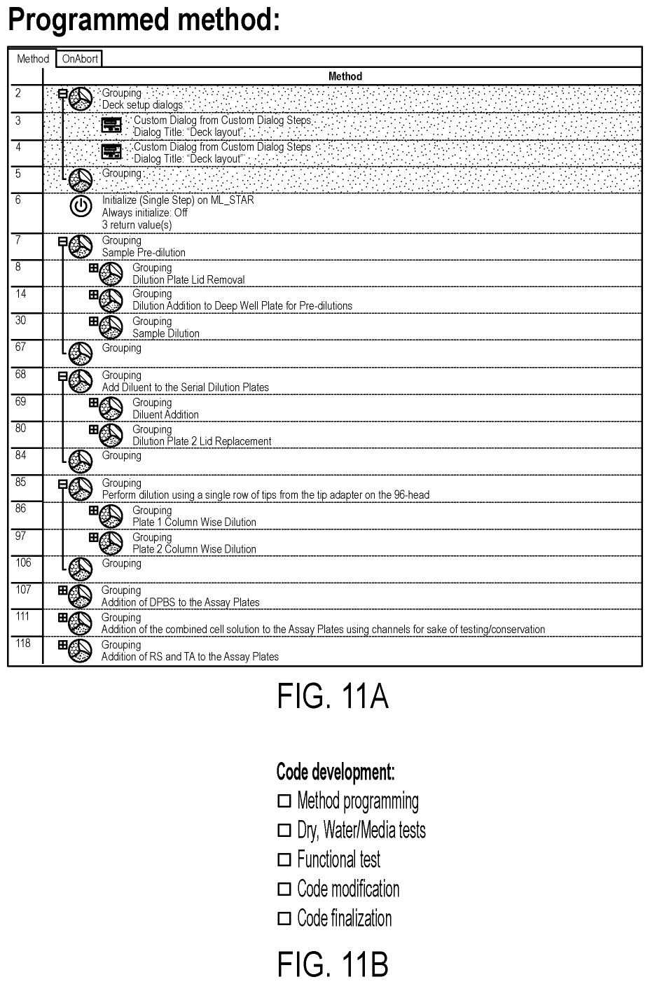

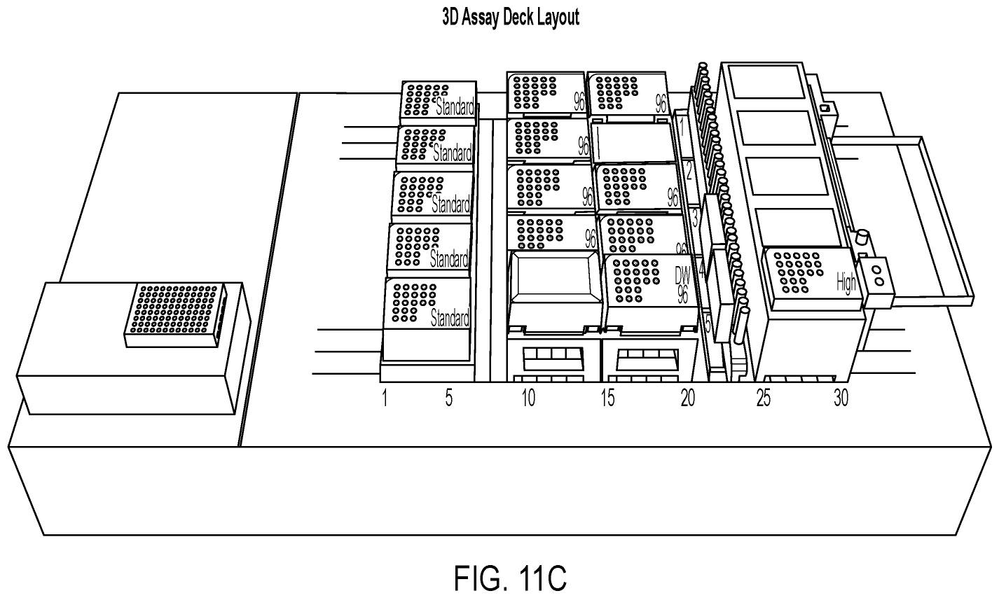

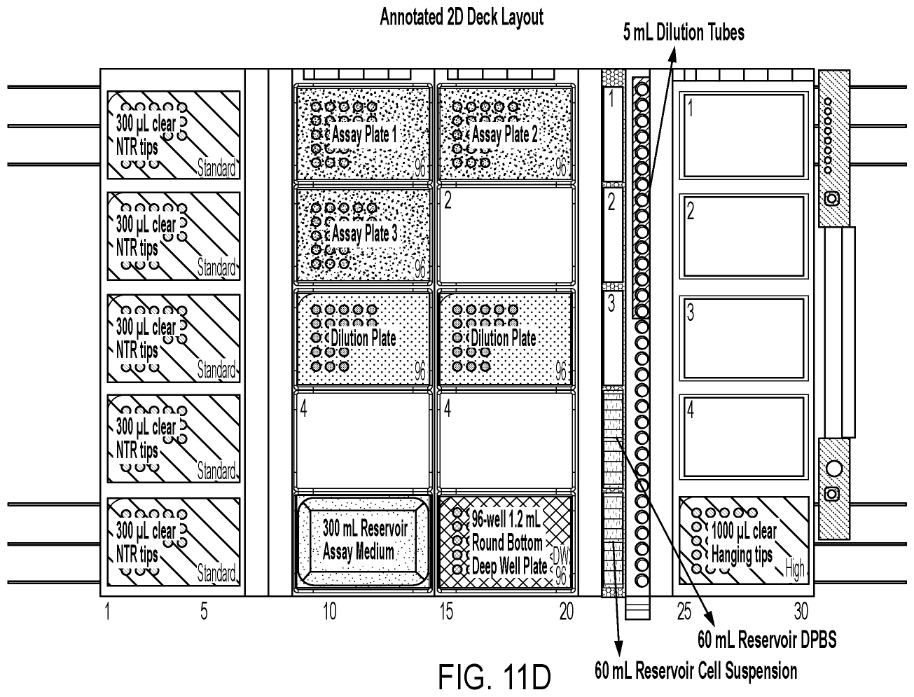

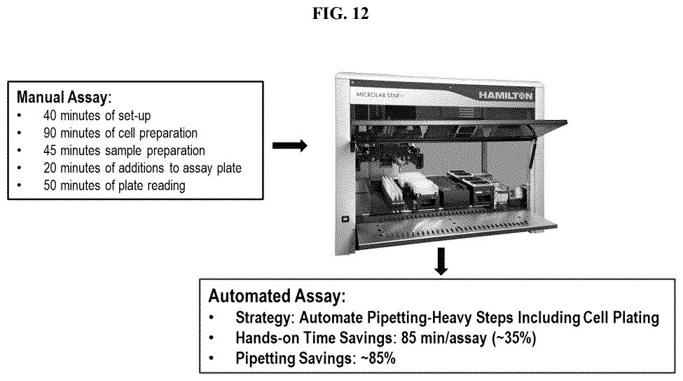

The anti-SARS-COV-2 neutralization assay was automated to execute the majority of procedures for Day 1 and Day 2 on the Integrated Laboratory Automated System (ILAS) platform. An overview of an exemplary workflow of an automated anti-SARS-COV2 neutralization assay, illustrating exemplary equipment integrated into the automated workflow using the methods and systems of the present invention, is illustrated in FIG. 7. An exemplary workflow of the automated method over the course of Day 1, Day 2 and Day 3 is illustrated in FIG. 8. Green solid boxes highlight the automated steps on the ILAS. The workflow of the assay is additionally illustrated in FIG. 9. The plate format for the assay is shown in FIG. 10. The layout of the automated method using the Hamilton STARlet is illustrated in FIG. 11. Benefits of the automated method to reduced operator time are illustrated in FIG. 12.

This study provided experimental evidence of potency measurement with reliable assay performance for the fully-automated anti-SARS-COV-2 neutralization assay. Samples used included a 122 mg/mL imdevimab sample and a 120 mg/mL casirivimab sample. The Day 1 cell seeding steps and the majority of Day 2 steps, including the majority of pre-dilution steps, for the anti-SARS-COV-2 neutralization assay were automated according to the methods and systems of the present invention. Only the Day 1 cell harvest, the Day 2 initial step of sample pre-dilution and pVSV-Luc-SARS-COV-2 pseudoparticle associated preparation, and the Day 3 One-Glo preparation were performed manually by analysts.

To demonstrate the efficacy of fully-automated assays of the present invention in comparison with assays performed manually by analysts, assay validity parameters such as system suitability and parallelism, assay performance characteristics such as accuracy and intermediate precision, and invalid rate—among other factors—were evaluated in the side-by-side comparison study. Each side-by-side comparison assay contained six plates, with three plates for the automated assay and three plates for manual assay. The plates were used for both reference standard (RS) and test article (TA) positions for studies of imdevimab or casirivimab, respectively. At the end, analysts read all six plates to generate results. Individual sample preparation was made for each RS and TA position of each plate. The validity criteria established for the manual assay were used to assess the validity of fully automated assays. One-way ANOVA analysis or equivalence test was used to assess if the fully automated anti-SARS-CoV-2 neutralization assay and manual assay were comparable or practically equivalent.

Three main parameters, the RS unconstrained R², the RS Max/Min Ratio and the Positive/Negative Control Ratio were used to evaluate the system suitability to ensure the method was performed on an appropriate system. The Max/Min Ratio is the ratio between the maximum average signal and minimum average signal of a reference standard. The Positive/Negative Control Ratio is the ratio between the average positive control signal and average negative control signal. These criteria were considered a requirement for the assay to be deemed valid.

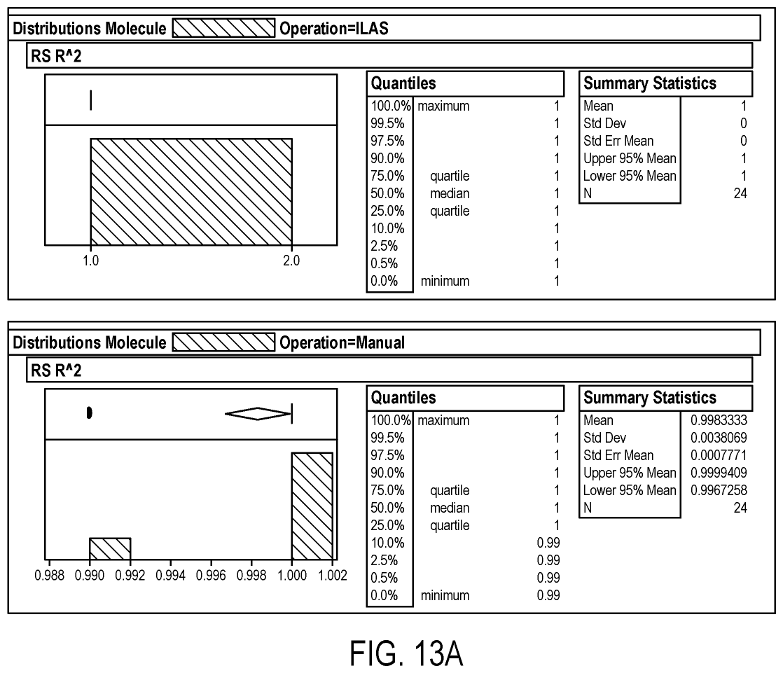

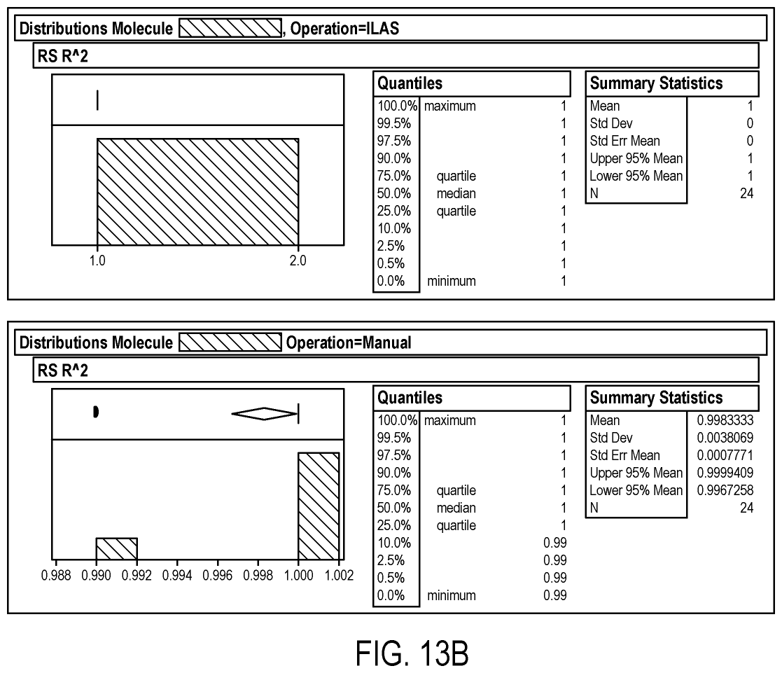

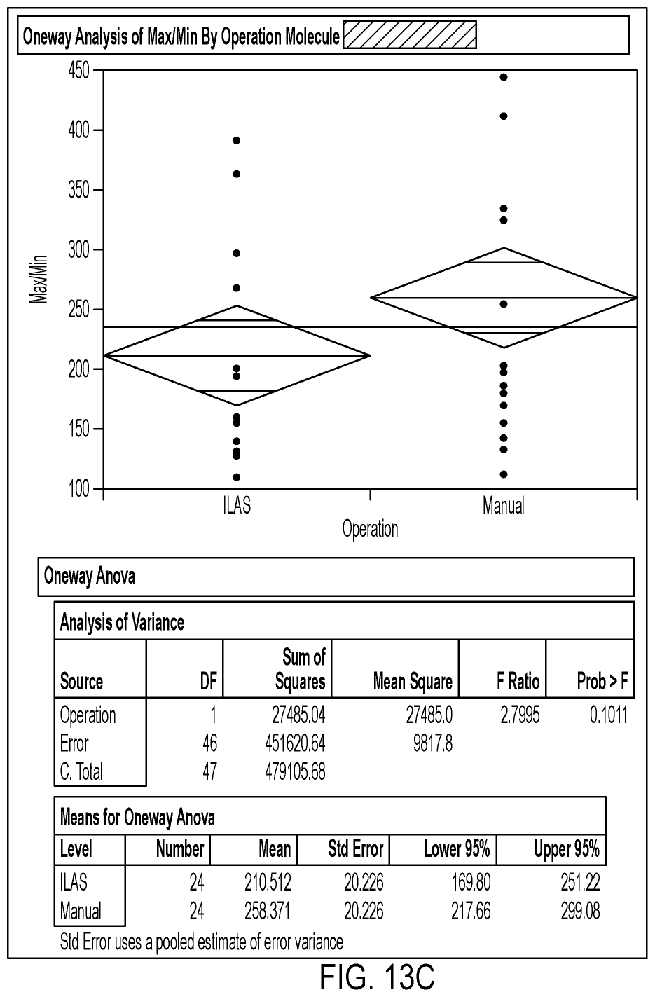

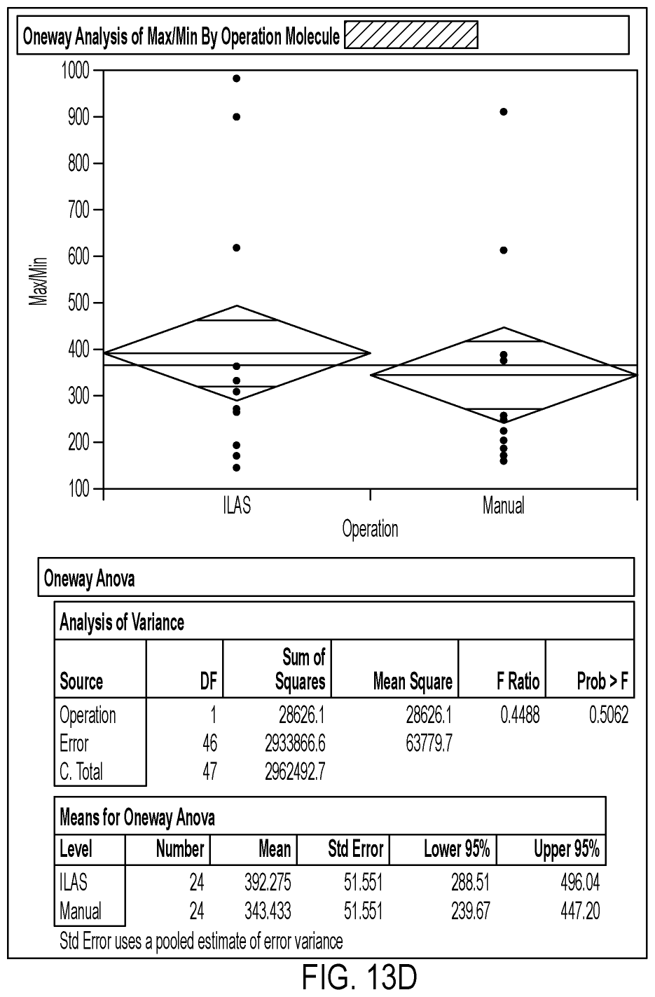

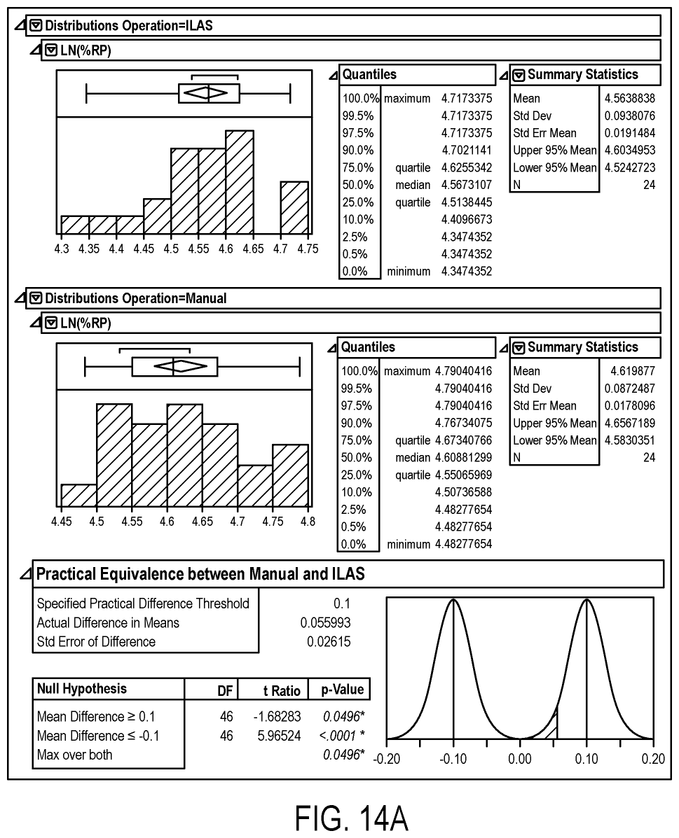

RS unconstrained R² were 1.00 in all fully-automated assays consistently with both tested antibodies, whereas RS unconstrained R² on the paired manual assays ranged from 0.99-1.00, as shown for imdevimab in FIG. 13A and casirivimab in FIG. 13B. These results demonstrated that automated assays had a comparable or better system suitability compared to manual assays.

One-way ANOVA analysis showed no significant difference on RS Max/Min Ratio between fully-automated and manual assays, as shown for imdevimab in FIG. 13C and casirivimab in FIG. 13D, suggesting a comparable assay background noise from assays performed on the ILAS to those performed manually.

Additionally, the geometric mean (or geomean) of Positive/Negative Control Ratios on fully-automated assays were 504.3 with imdevimab and 433.0 with casirivimab. These data met ≥15 acceptance criteria. The geometric mean is calculated as follows:

$${e}^{∧} ( \frac{{\sum }_{i = I}^{n} ln {x}_{i}}{n} )$$

where xᵢ is each plate % relative potency value per TA and n is the number of plates.

In conclusion, these results demonstrated that the automated assay behaved in a manner consistent with or better than the manual assay.

RS IC50 values were also compared between the automated assay and the manual assay using an equivalence test. An IC50, or half maximal inhibitory concentration, is a measure of the effectiveness of a substance in inhibiting a specific biological or biochemical function. In this assay, this quantitative measure indicates how much anti-SARS-COV-2 antibody is needed to inhibit the binding of the pseudoparticle spike protein to the ACE2 receptor on the Vero cells. The IC50 represents the concentration of drug that is required for 50% inhibition of binding.

A practical difference threshold setting from −2.000 ng/ml to 2.000 ng/ml for imdevimab and from −1.650 ng/mL to 1.650 ng/mL for casirivimab were selected based on analyst variance. The 95% confidence interval (CI) of RS IC50 difference between manual and automated assays from Student's t-test was used to demonstrate equivalence. 95% CI of difference needs to fall within the practical difference threshold as an indicator of equivalence.

The 95% CI for RS IC50 manual-automated difference and practical difference threshold is shown in Table 1. All 95% CI of RS IC50 difference between the manual and automated assay fell within the practical difference. These data suggest that the RS IC50 measured between the manual assay and the automated assay are equivalent.

TABLE 1
Summary of RS IC50 difference between
manual and automated assays

| | Practical difference | 95% CI for manual- | |
|---|---|---|---|
| Molecule | threshold | automation difference | Status |
| Imdevimab | −2.000 ng/mL | −0.643 ng/mL | Equivalent |
| | to +2.000 ng/mL | to +1.367 ng/mL | |
| Casirivimab | −1.650 ng/mL | −12.30 ng/mL | Equivalent |
| | to +1.650 ng/mL | to −0.075 ng/mL | |

As a prerequisite to the calculation of relative potency, parallelism parameters were assessed to ensure similarity between TA and RS. When RS and TA preparations are similar and assay responses are plotted against the concentration in log scale, the resulting constrained TA curve should be the same as the RS curve but shifted horizontally by an amount equivalent to the logarithm of the relative potency estimates. If the two curves are not similar enough, an accurate relative potency cannot be measured, and thus its use as a comparison of TA to RS is no longer meaningful.

To evaluate if the fully-automated anti-SARS-COV-2 neutralization assay can be used to demonstrate the similarity of TA to RS, parameters (i.e. UAR, SR and A*B ratio) used for a parallelism assessment from the automated assay were evaluated against the criteria established from the manual assay. The Upper Asymptote Ratio (UAR, or A Ratio) is the ratio between the TA upper asymptote and the RS upper asymptote. The Slope Ratio (SR, or B Ratio) is the ratio between the slope of the TA response curve and the slope of the RS response curve. The A*B Ratio is the ratio of the upper asymptote times slope between the TA and RS, and is equal to the UAR (A Ratio) times the SR (B Ratio).

The averages of UAR, SR and A*B ratio along with their 95%/95% tolerance intervals (95/95 TI) were calculated for both fully-automated assays and manual assays and are summarized in Table 2. The averages and their 95/95 TI of UAR, A*B ratio and SR of fully-automated assays all met the acceptance criteria established by the manual assay, indicating that the fully-automated anti-SARS-COV-2 neutralization assays were able to release product meeting the parallelism criteria established from the manual assay. Additionally, the lower 95% TI of A*B ratio in manual assays (0.73) was lower than the acceptance criteria (0.75-1.30), whereas the 95/95 TI from side-by-side fully-automated assays (0.80-1.21) were well within the acceptance criteria, suggesting a more stable performance from the automated assays than from manual assays.

TABLE 2
Summary of parallelism parameter results

| | | | Manual | | | Automated | | |
|---|---|---|---|---|---|---|---|---|
| | | Acceptance | | 95/95 | | | 95/95 | |
| Molecule | Parameters | Criteria | Geomean | TI | Status | Geomean | TI | Status |
| Imdevimab | UAR | 0.90-1.10 | 0.99 | 0.91-1.08 | Met | 0.99 | 0.94-1.05 | Met |
| | A*B Ratio | 0.75-1.30 | 1.01 | 0.86-1.17 | Met | 0.98 | 0.83-1.13 | Met |
| | SR | 0.75-1.30 | 1.02 | 0.84-1.20 | Met | 1.00 | 0.82-1.16 | Met |
| Casirivimab | UAR | 0.90-1.10 | 0.98 | 0.92-1.04 | Met | 1.00 | 0.99-1.01 | Met |
| | A*B Ratio | 0.75-1.30 | 1.00 | 0.73-1.28 | Not Met | 1.00 | 0.80-1.21 | Met |
| | SR | 0.75-1.30 | 1.02 | 0.82-1.16 | Met | 1.00 | 0.78-1.24 | Met |

To compare the efficacy of the fully-automated assay against the manual assay, reportable relative potency (% RP) was assessed. A % relative potency (or PLA potency) for each TA is calculated from the quotient of IC50 values determined from the parallel line analysis (PLA) curves, as follows:

PLA Potency=constrained IC50(RS)/constrained IC50(TA)

Plate % relative potency=PLA Potency*100%

The geometric mean value of the three plate relative potencies is the reportable relative potency (% RP). In each plate, two position-specific % RP and an overall % RP across all TA positions were generated. The overall % RP was compared between the automated assay and the manual assay followed by a position-specific % RP comparison for position bias evaluation.

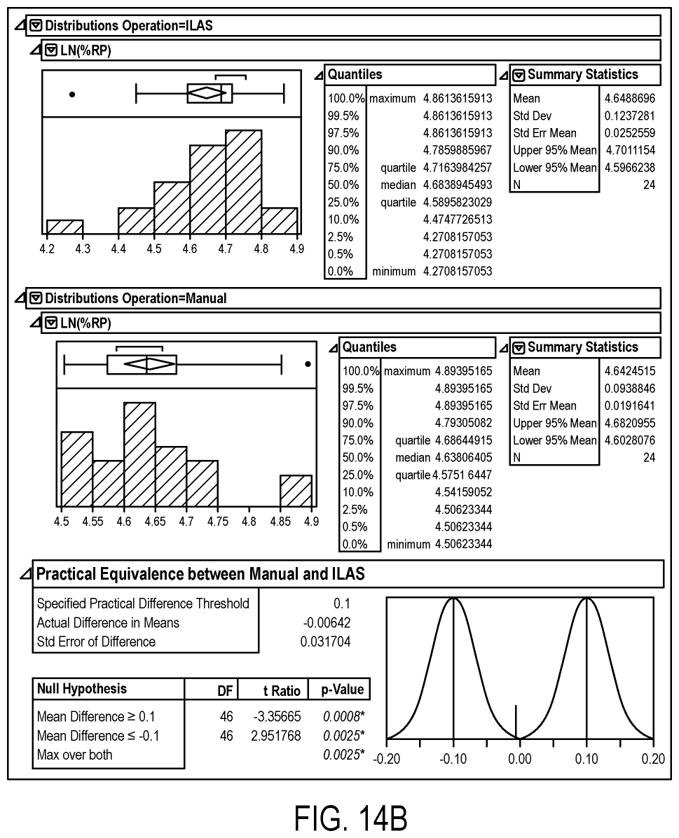

To compare if the reportable potency obtained from fully-automated and manual assays are equivalent, an equivalence test with an equivalence bound of 90-110% was performed based on the intrinsic assay variability to compare the manual assay with the automated assay, as shown for imdevimab in FIG. 14A and casirivimab in FIG. 14B. Based on the data, the potency measurements of automated assays and manual assays are practically equivalent for both imdevimab and casirivimab.

As shown in Table 3, the geometric mean of imdevimab reportable potency was 96%, and the geometric mean of casirivimab reportable potency was 105%. All reportable potencies were well within the empirical 80-125% acceptance range used for assay validation. Additionally, the 95% CI of the reportable potency with each molecule was well within the 80-125% acceptance range. Therefore, the automated anti-SARS-COV-2 neutralization assay provides a good reportable potency.

TABLE 3
Reportable potency of automated assays

| | Reportable | | Empirical | |
|---|---|---|---|---|
| Molecule | potency | 95% CI | acceptable range | Status |
| Imdevimab | 96% | 93%-100% | 80%-125% | Met |
| Casirivimab | 105% | 101%-110% | | Met |

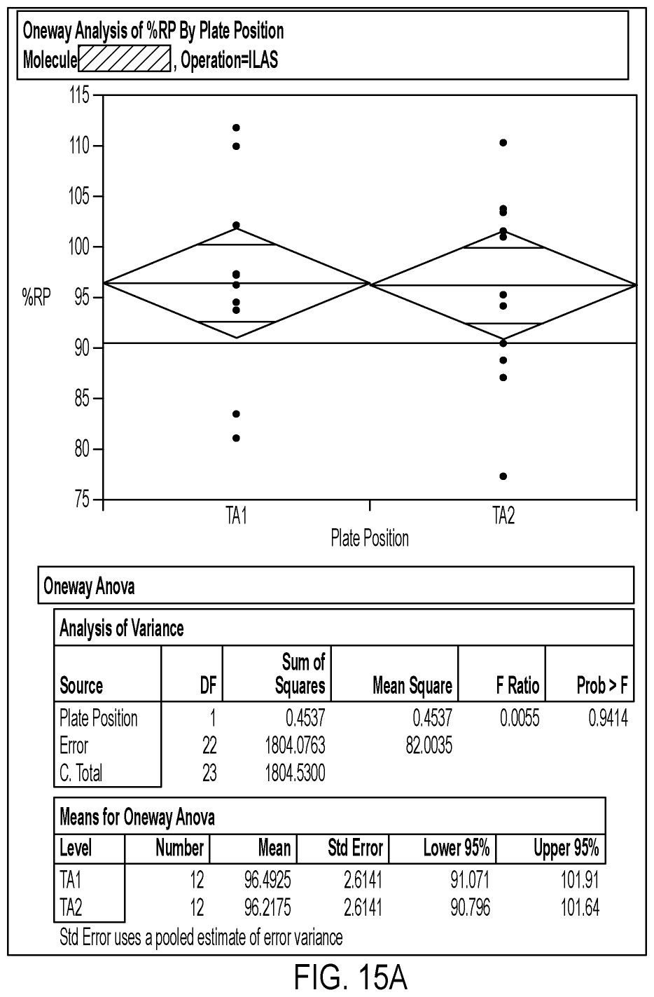

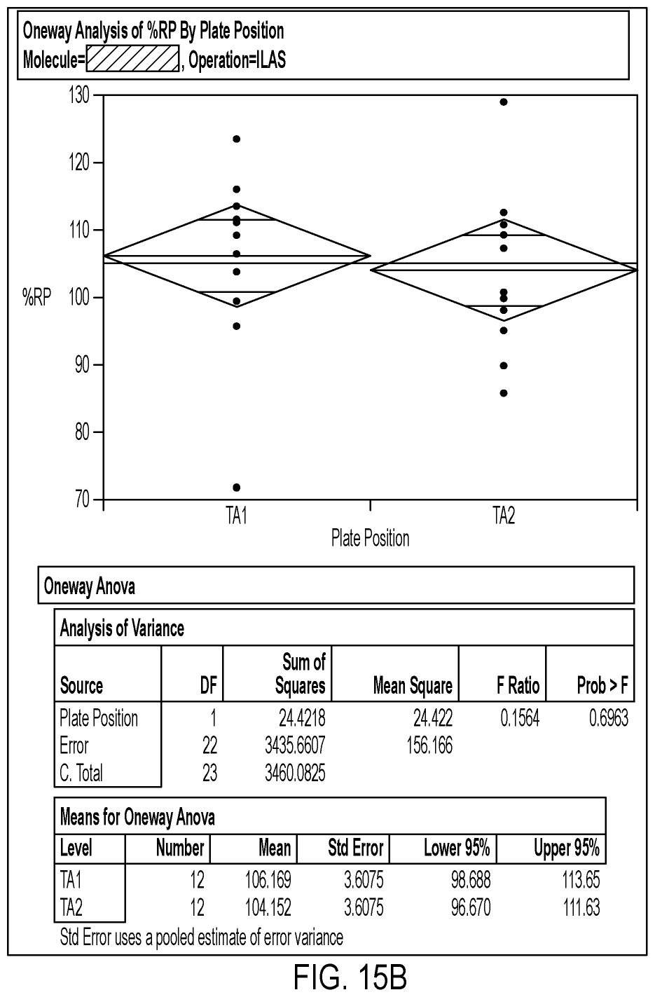

Next, to evaluate if the automated assay displays position bias, the % RP of each TA position from the automated assay was assessed and compared. One-way ANOVA analysis demonstrated that there is no significant difference (p >0.05) between the two TA positions in the automated assay with imdevimab and casirivimab regarding potency measurement, indicating that the automated assay has no position bias, as shown in FIG. 15A for imdevimab and FIG. 15B for casirivimab.

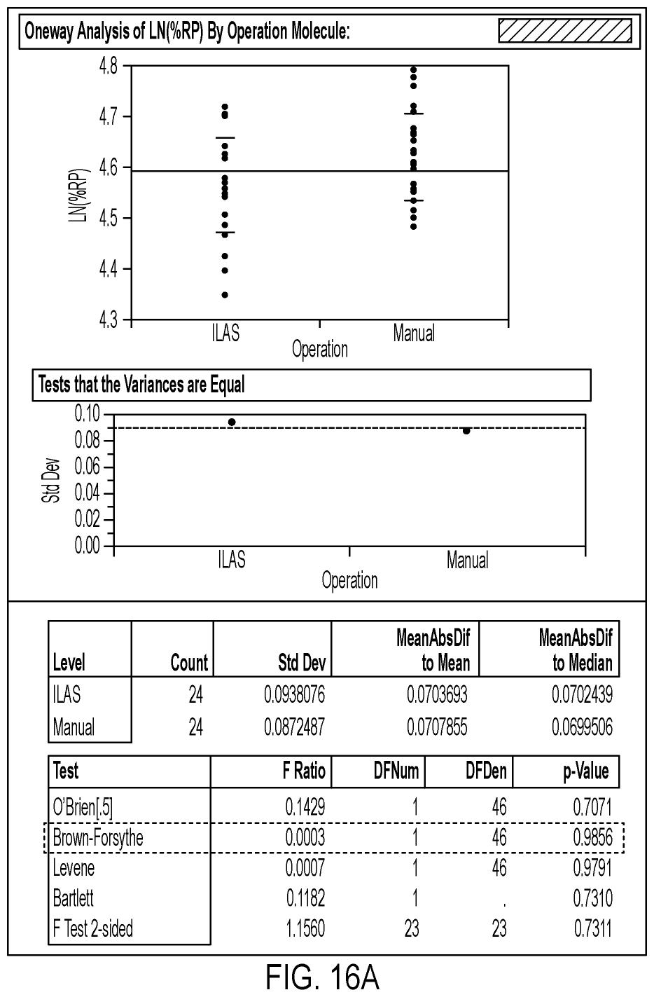

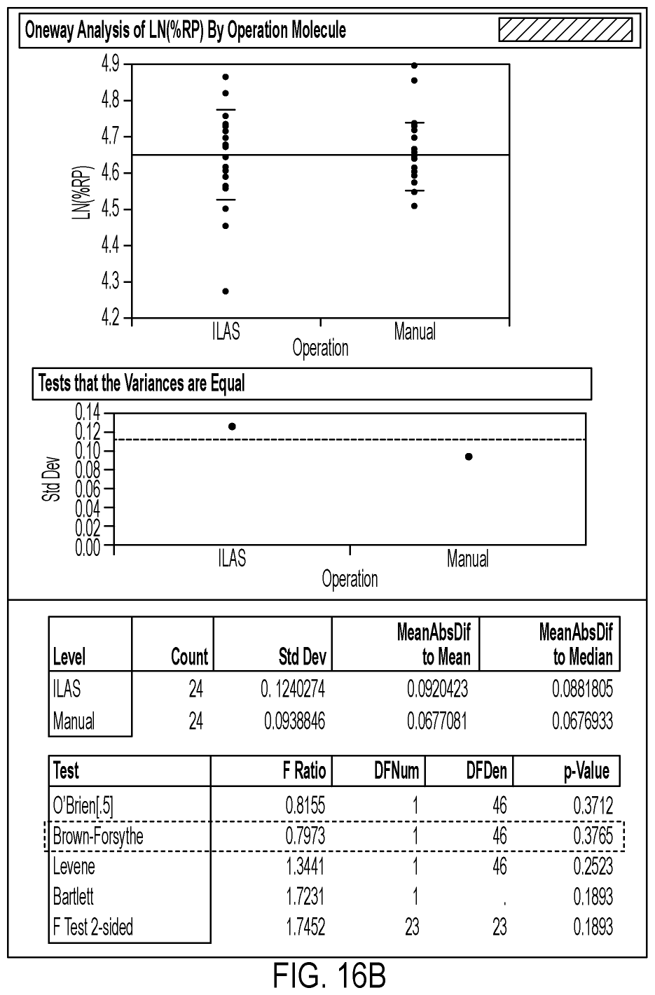

To evaluate if a fully-automated assay performs with equal or better precision than a manual assay, a Brown-Forsythe test was run to compare assay variability. The result demonstrated that there was no significant difference (p >0.05) for variance comparison between manual and fully-automated assays with both imdevimab, as shown in FIG. 16A, and casirivimab, as shown in FIG. 16B, suggesting that the fully-automated assay is equally precise as a manual assay for potency measurement.

Additionally, the intermediate precision (using percent geometric coefficient of variation, or % GCV) of automated assays and manual assays were calculated quantitatively for both molecules, as shown in Table 4. The intermediate precision of fully-automated assays ranged from 4% to 7%, and the upper 95% CI ranged from 8% to 14%, which are well within the empirical acceptance criteria of ≤30% used in assay validation, indicating that the automated assay has a well-controlled assay variance and is suitable for use.

TABLE 4
Intermediate precision of fully-automated assays and manual assays

| | Manual | | Starlet | | | |
|---|---|---|---|---|---|---|
| | | Upper 95% | | Upper 95% | Empirical | |
| Molecule | % GCV | CI Limit | % GCV | CI Limit | Acceptance Range | Status |
| Imdevimab | 3 | 6 | 4 | 8 | ≤ 30% | Met |
| Casirivimab | 7 | 13 | 7 | 14 | | Met |

In a side-by-side comparison study, the assay invalid rate was also monitored as part of the assay performance evaluation. The invalid rate was calculated by the amount of invalid TA dose-response curves divided by the total (valid and invalid) TA curves. Table 5 summarizes the invalid rate of automated and manual assays for both molecules imdevimab and casirivimab. Neither the fully-automated assay nor the manual assay failed, indicating a good and stable overall performance of the automated assay.

TABLE 5
Summary of invalid rate

| | Manual | | Starlet | |
|---|---|---|---|---|
| | | Total | | Total |
| Molecule | Invalid Curve | Curve | Invalid Curve | Curve |
| Imdevimab | 0 | 24 | 0 | 24 |
| | Overall | 0% | Overall | 0% |
| | Invalid Rate | | Invalid Rate | |
| Casirivimab | 0 | 24 | 0 | 24 |
| | Overall | 0% | Overall | 0% |
| | Invalid Rate | | Invalid Rate | |

In the side-by-side comparison study described above, system suitability, parallelism (or sample suitability), potency measurement, and assay invalid rate were evaluated using either one-way ANOVA analysis or equivalence test, which showed that automated assays were comparable to or better than those run manually by analysts.

In conclusion, automated GMP-compliant assay methods and systems of the present invention proved to be comparable to paired manual assays when used to conduct an exemplary anti-SARS-COV-2 neutralization assay.

### Example 3. Linearity of the Automated GMP-Compliant Assays

In order to further validate the automated GMP-compliant assay methods and systems of the present invention, a linearity study of an automated assay for anti-SARS-COV-2 neutralization with imdevimab and casirivimab was conducted.

Automated assays were performed at three target potency levels: 50%, 100% and 160%, which were prepared by diluting imdevimab or casirivimab in assay media on the day the assay was to be performed. For each molecule, two potency levels were run as two TAs on each plate. Three analysts ran a set of two plates with two individual dilution preparations made for each potency level across three plates for imdevimab; two analysts ran a set of three plates with two dilution preparations made for each potency level across three plates for casirivimab; resulting in twelve potency determinations (i.e. four reportable potency values) generated at each level with imdevimab and casirivimab. Each assay was run using fully-automated steps on the ILAS except for the steps of cell harvest on Day 1, initial sample preparation, VLP, positive and negative VLP control preparation on Day 2, and One-Glo preparation on Day 3. From this linearity study, accuracy and intermediate precision of the assay were assessed.

As described above, the targeted 50%, 100%, and 160% potency levels were tested by three analysts with imdevimab and two analysts with casirivimab. The accuracy (% recovery) at each potency level was determined by comparing the geometric mean of the measured relative potency values for all assays performed at each target potency level to the expected % RP. Specifically, the accuracy (% recovery) was calculated by dividing the observed % geometric mean relative potency (% GMRP) by the expected % RP multiplied by 100. Accuracy data are summarized in Table 6.

TABLE 6
Accuracy data of automated assay on ILAS

| | Expected | Observed | Accuracy | | Acceptance | |
|---|---|---|---|---|---|---|
| Molecule | % RP | % GMRP | (% Recovery) | 95% CI | Range | Status |
| Imdevimab | 50 | 50 | 100% | 91%-111% | 80%-125% | Met |
| | 100 | 102 | 102% | 90%-116% | | Met |
| | 160 | 166 | 104% | 93%-116% | | Met |
| | Overall % Accuracy | | 101% | 96%-107% | | Met |
| Casirivimab | 50 | 50 | 101% | 91%-111% | | Met |
| | 100 | 97 | 97% | 91%-103% | | Met |
| | 160 | 166 | 104% | 93%-116% | | Met |

| | | | |
|---|---|---|---|
| Overall % Accuracy | 100% | 96%-105% | Met |

As shown in Table 6, the average accuracy at each target potency level with imdevimab ranged from 100% to 104% and the overall accuracy was 101%. The average accuracy at each target potency level with casirivimab ranged from 97% to 104% and the overall accuracy was 100%. The accuracy for these tests was well within the 80-125% acceptance range used for assay validation. Additionally, the 95% CI of the average accuracy at each potency level and overall accuracy with both imdevimab and casirivimab are within the 80-125% acceptance range. Therefore, the accuracy of a fully-automated anti-SARS-COV-2 neutralization assay was well within acceptable ranges.

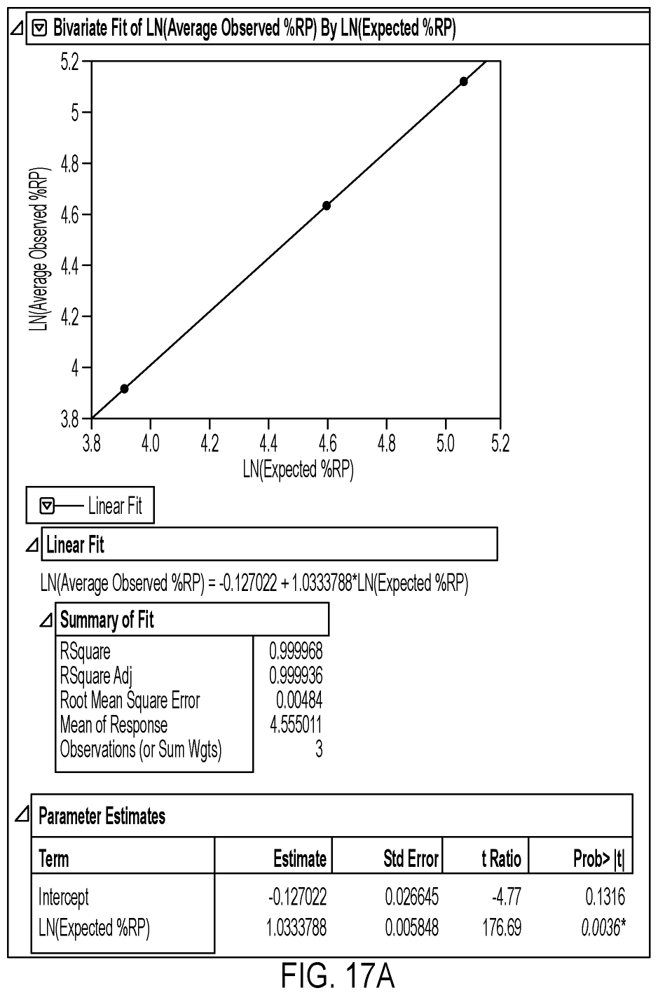

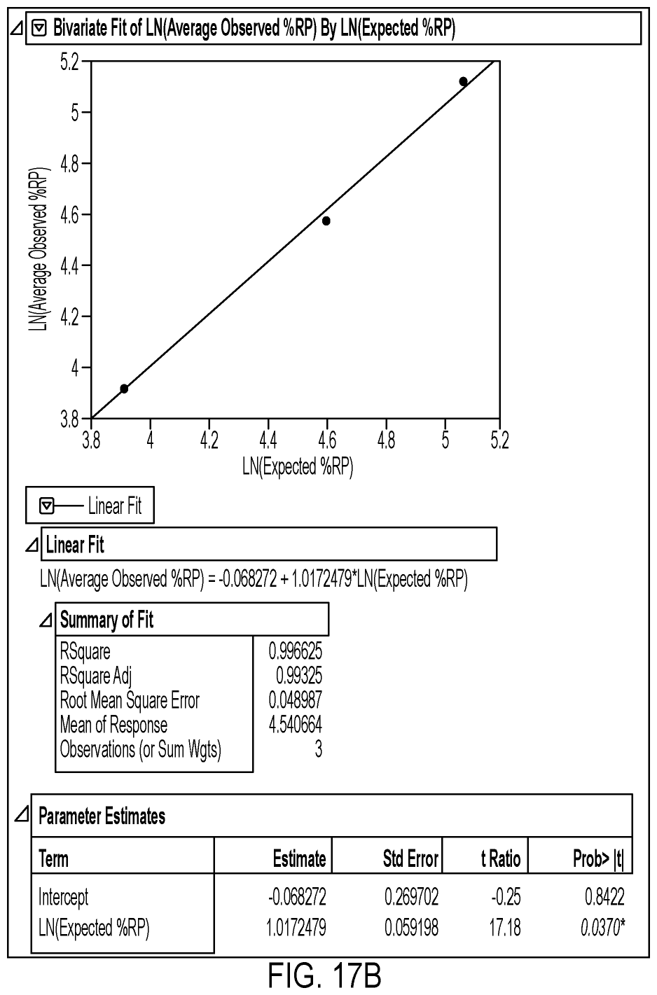

Linearity was determined using the three potency levels. The overall linearity was obtained by determining the linear fit of the average of the log-transformed relative potency values at each potency level across all analysts (n=4 for each potency level), as shown in FIG. 17A for imdevimab and FIG. 17B for casirivimab. The R² value for the average of all data was 1.0000 with imdevimab and 0.9966 with casirivimab, which was greater than the acceptance criterion of 0.98 used in assay validation, indicating a good linear fit of the model with data generated from the automated method. For the overall linearity plot with imdevimab, the y-intercept was −0.127022 and the slope was 1.0333788. For the overall linearity plot with casirivimab, the y-intercept was −0.068272 and the slope was 1.0172479.

The % GCV was calculated for the 4 values at each potency level from all valid assays. The intermediate precision of all potency levels from assays with imdevimab ranged from 7% to 8%, with an overall precision of 8%, and 95% CI ranged from 20% to 27%, with an overall upper 95% CI of 15%. The intermediate precision of all potency levels from assays with casirivimab ranged from 4% to 7%, with an overall precision of 7%, and the 95% CI ranged from 12% to 23%, with an overall upper 95% CI of 12%. All intermediate precisions met the assay validation acceptance criterion of ≤30%, indicating that the variance of the automated assay is within the normal range of assay variation, as shown in Tables 7 and 8.

TABLE 7
Intermediate precision data of automated assay for imdevimab

| Expected | Observed | % | Upper | Acceptance | |
|---|---|---|---|---|---|
| % RP | % GMRP | GCV | 95% CI | Range | Status |
| 50 | 50 | 7 | 20 | ≤30% | Met |
| 100 | 102 | 8 | 27 | | Met |
| 160 | 166 | 7 | 22 | | Met |

| | | | |
|---|---|---|---|
| Overall % Accuracy | 8 | 15 | Met |

TABLE 8
Intermediate precision data of automated assay for casirivimab

| Expected | Observed | % | Upper | Acceptance | |
|---|---|---|---|---|---|
| % RP | % GMRP | GCV | 95% CI | Range | Status |
| 50 | 50 | 6 | 20 | ≤30% | Met |
| 100 | 97 | 4 | 12 | | Met |
| 160 | 166 | 7 | 23 | | Met |

| | | | |
|---|---|---|---|
| Overall % Accuracy | 7 | 12 | Met |

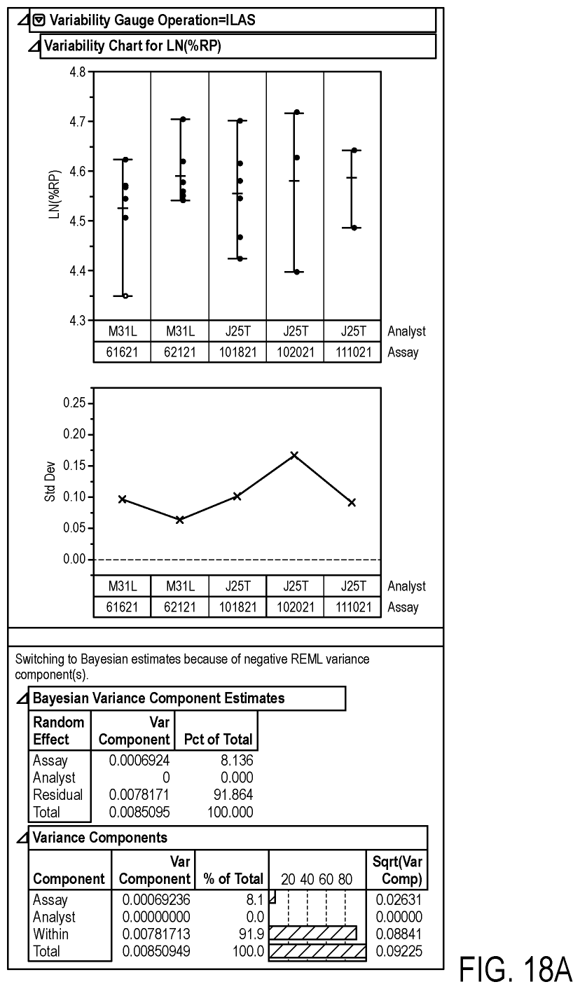

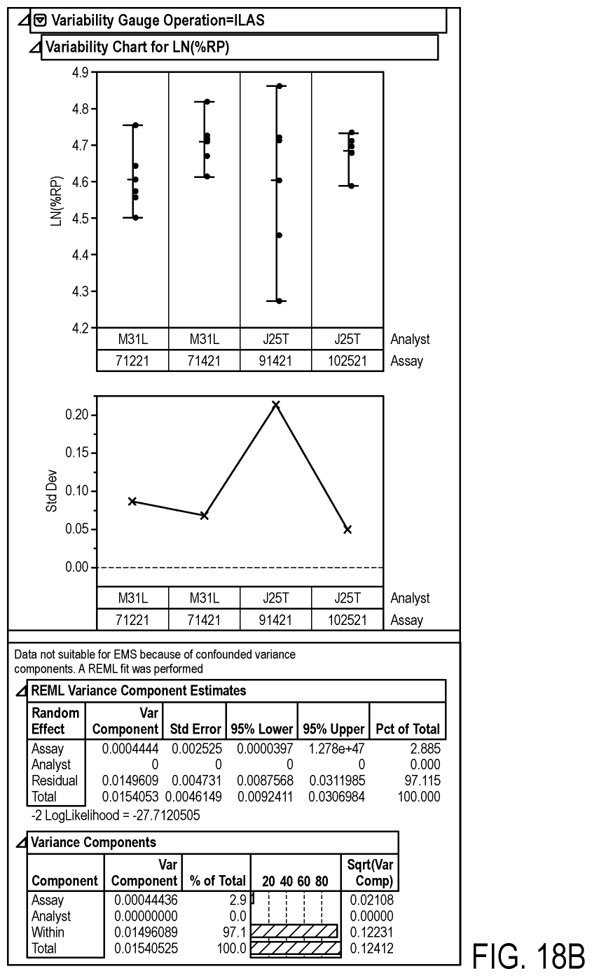

Additionally, a variance component analysis was performed to estimate the sources of variance, where assay day and analyst were analyzed in assays with either imdevimab or casirivimab, as shown in FIG. 18A and FIG. 18B respectively. The log-transformed % RP displays a relatively consistent spread with a similar standard deviation across analysts and assay day. The variance component in assays with imdevimab showed that 8.1% and 0% of the variation came from assay day and analyst, respectively. The variance component in assays with casirivimab showed that 2.9% and 0% of the variation came from assay day and analyst, respectively. These data suggest that neither analyst nor assay day are major contributors to the overall variability.

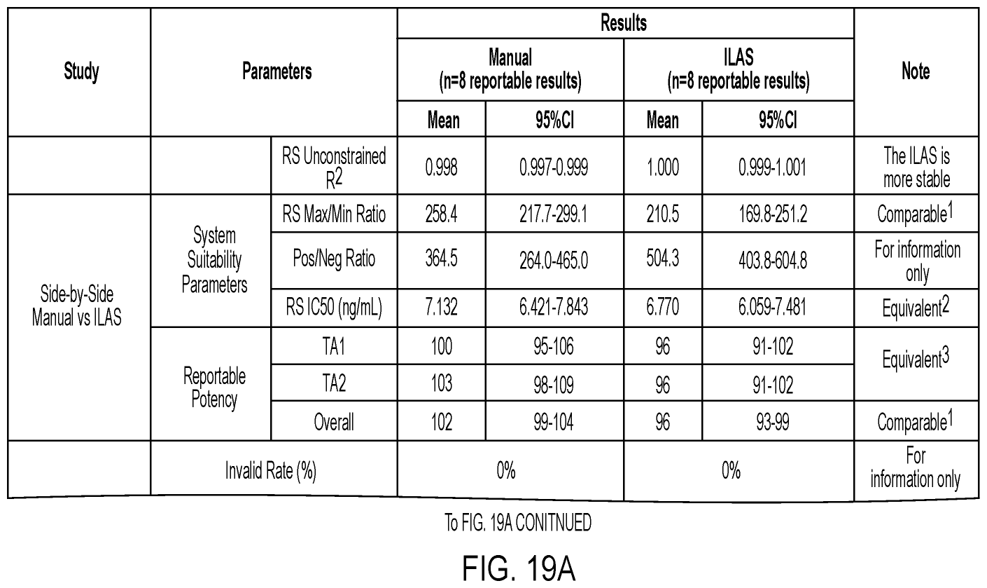

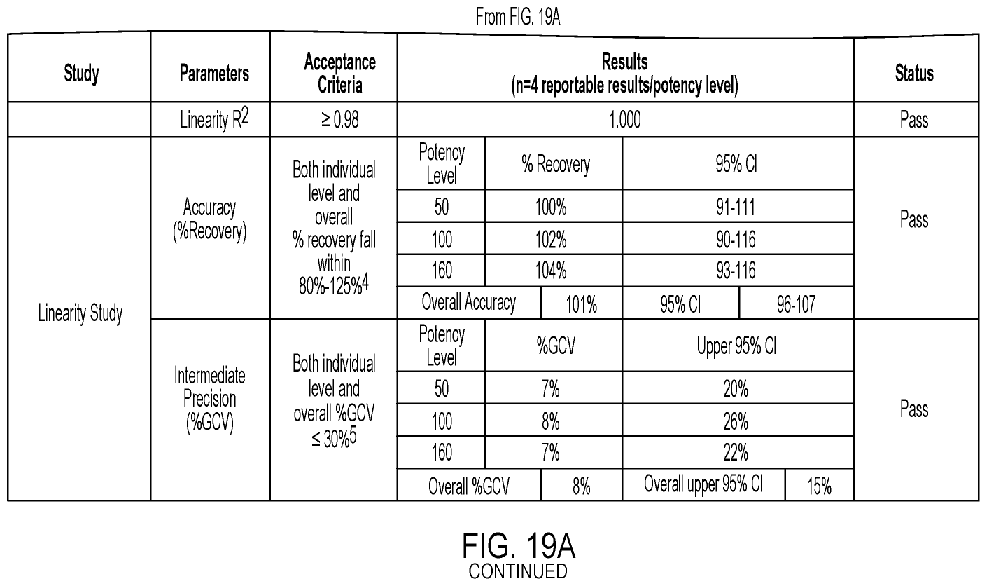

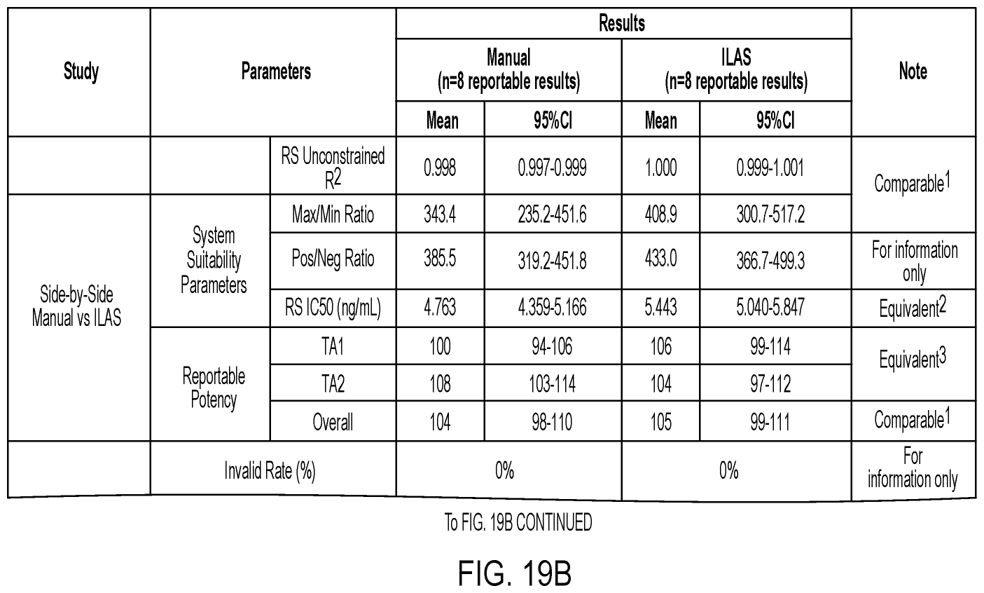

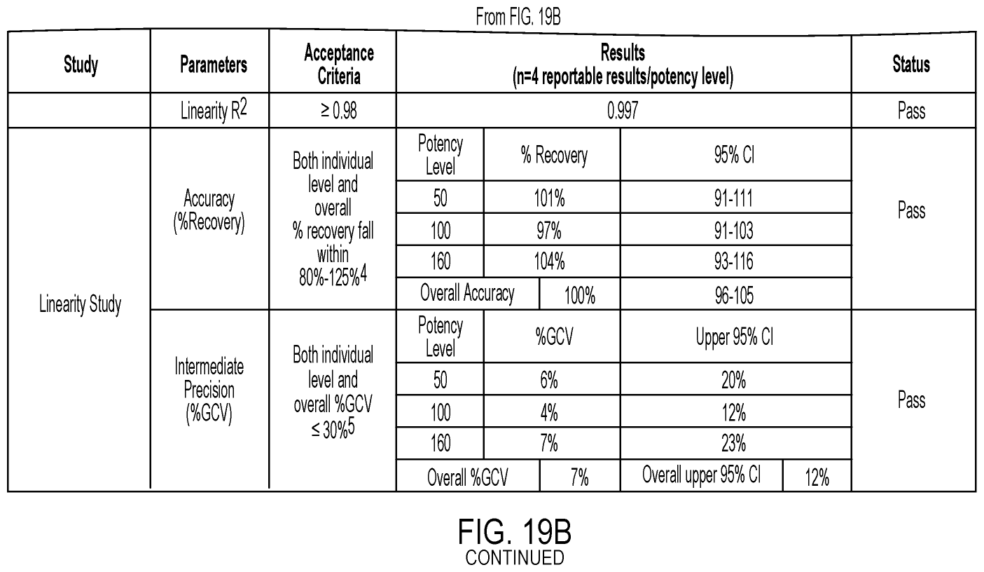

Summaries of the side-by-side tests and linearity tests using automated GMP-compliant methods and systems of the present invention are provided for imdevimab in FIG. 19A and casirivimab in FIG. 19B. In conclusion, automated GMP-compliant assay methods and systems of the present invention achieved satisfactory accuracy and precision when used to conduct an exemplary anti-SARS-COV-2 neutralization assay.

What is claimed is:

1. An automated GMP-compliant method for conducting an assay, the method comprising:
developing an assay protocol for a GMP-compliant assay;
storing the assay protocol to include any changes and record of associated usage data, wherein any change to the protocol is stored and tracked;
communicating the assay protocol to a first secure computer system in accordance with the assay protocol;
subjecting at least one sample to the assay protocol executed by the first computer system, wherein the first computer system causes an automated operation of the assay protocol on the sample;
collecting data associated with the at least one sample subjected to the assay protocol as part of the automated operation of the assay protocol on the sample; and
generating a GMP-compliant dataset from the collected data as part of the automated operation of the assay protocol on the sample, wherein the GMP-compliant dataset includes an audit trail of the dataset, identification of location data for the at least one sample throughout execution of the assay protocol, and a record of any changes to the assay protocol, software and/or equipment controlled by the first computer system.

2. The method of claim 1, wherein the assay is a bioassay.

3. The method of claim 1, wherein the protocol is optimized using data collected from at least one sample subjected to the protocol.

4. The method of claim 1, wherein the protocol is protected by a password.

5. The method of claim 1, wherein the protocol is subjected to quality control review between 1 and 31 times per month, between 1 and 10 times per month, between 1 and 7 times per week, 1 time per week, 2 times per week, or 3 times per week.

6. The method of claim 1, wherein the first computer system executes the protocol using a scheduling software.

7. The method of claim 6, wherein the scheduling software is Cellario.

8. The method of claim 1, wherein an automated operation of the protocol includes a robotic arm.

9. The method of claim 8, wherein the robotic arm is an ACell robotic arm.

10. The method of claim 1, wherein an automated operation of the protocol includes at least one liquid handler and/or reagent dispenser.

11. The method of claim 10, wherein the at least one liquid handler and/or reagent dispenser is a Hamilton STARlet and/or a Multidrop Combi Reagent Dispenser.

12. The method of claim 1, wherein the at least one sample is selected from a group consisting of cell culture fluid, harvested cell culture fluid, filtrate, chromatography eluate, drug substance, and drug product.

13. The method of claim 1, wherein the at least one sample includes at least one therapeutic protein, wherein said protein is selected from a group consisting of an antibody, a monoclonal antibody, a bispecific antibody, a fusion protein, an antibody-drug conjugate, a receptor, and an antibody fragment.

14. The method of claim 13, wherein the at least one therapeutic protein is imdevimab or casirivimab.

15. The method of claim 13, wherein the at least one therapeutic protein is dupilumab.

16. The method of claim 1, wherein collecting data comprises subjecting the at least one sample to at least one measurement.

17. The method of claim 16, wherein said at least one measurement is selected from a group consisting of spectrophotometry, ultraviolet detection, fluorescence detection, luminescence detection, radioactivity detection, Raman spectroscopy, mass spectrometry, biolayer interferometry, surface plasmon resonance, and absorbance detection.

18. The method of claim 1, wherein said at least one sample is contained in a microplate.

19. The method of claim 1, wherein collecting data includes using at least one data analysis software.

20. The method of claim 19, wherein said at least one data analysis software is SoftMax.

21. The method of claim 1, wherein said dataset includes a unique identifier for said at least one sample.

22. The method of claim 1, wherein said dataset includes a unique identifier for a container containing said at least one sample.

23. The method of claim 1, further comprising generating a unique identifier for a container containing said at least one sample.

24. The method of claim 23, wherein said unique identifier is a barcode.

25. The method of claim 24, wherein said barcode is generated using Sci-Print MP2+.

26. The method of claim 24, wherein said container is labeled with said barcode using Sci-Print MP2+.

27. The method of claim 24, further comprising scanning said barcode at a critical step of subjecting said at least one sample to said assay protocol to generate location data for said at least one sample.

28. The method of claim 1, wherein said assay is a cell-based assay.

29. An automated system for conducting a GMP-compliant assay, comprising:
a computer system, wherein the computer system stores an assay protocol for a GMP-compliant assay, and wherein the computer system is capable of collecting data associated with at least one sample subjected to the assay protocol and producing a GMP-compliant dataset; and
at least one automated equipment capable of subjecting at least one sample to an assay protocol, wherein the computer system causes automated operation of the at least one automated equipment,
wherein the GMP-compliant dataset includes an audit trail of the dataset, identification of location data for the at least one sample throughout execution of the assay protocol, and a record of any changes to the assay protocol, software and/or equipment controlled by the secure computer system.

30. The system of claim 29, wherein the assay is a bioassay.

31. The system of claim 29, wherein the protocol is created using Cellario.

32. The system of claim 29, wherein the protocol is optimized using data collected from at least one sample subjected to the protocol.

33. The system of claim 29, wherein the protocol is protected by a password.

34. The system of claim 29, wherein the protocol is subjected to quality control review between 1 and 31 times per month, between 1 and 10 times per month, between 1 and 7 times per week, 1 time per week, 2 times per week, or 3 times per week.

35. The system of claim 29, wherein the computer system causes automated operation of the at least one automated equipment using a scheduling software.

36. The system of claim 35, wherein the scheduling software is Cellario.

37. The system of claim 29, wherein the at least one automated equipment includes a robotic arm.

38. The system of claim 37, wherein the robotic arm is an ACell robotic arm.

39. The system of claim 29, wherein the at least one automated equipment includes at least one liquid handler and/or reagent dispenser.

40. The system of claim 39, wherein the at least one liquid handler and/or reagent dispenser is a Hamilton STARlet and/or a Multidrop Combi Reagent Dispenser.

41. The system of claim 29, wherein the at least one sample is selected from a group consisting of cell culture fluid, harvested cell culture fluid, filtrate, chromatography eluate, drug substance, and drug product.

42. The system of claim 29, wherein the at least one sample includes at least one therapeutic protein, wherein said protein is selected from a group consisting of an antibody, a monoclonal antibody, a bispecific antibody, a fusion protein, an antibody-drug conjugate, a receptor, and an antibody fragment.

43. The system of claim 42, wherein the at least one therapeutic protein is imdevimab or casirivimab.

44. The system of claim 42, wherein the at least one therapeutic protein is dupilumab.

45. The system of claim 29, wherein collecting data comprises subjecting the at least one sample to at least one measurement.

46. The system of claim 45, wherein said at least one measurement is selected from a group consisting of spectrophotometry, ultraviolet detection, fluorescence detection, luminescence detection, radioactivity detection, Raman spectroscopy, mass spectrometry, biolayer interferometry, surface plasmon resonance, and absorbance detection.

47. The system of claim 29, wherein said at least one sample is contained in a microplate.

48. The system of claim 29, wherein collecting data includes using at least one data analysis software.

49. The system of claim 48, wherein said at least one data analysis software is SoftMax.

50. The system of claim 29, wherein said dataset includes a unique identifier for said at least one sample.

51. The system of claim 29, wherein said dataset includes a unique identifier for a container containing said at least one sample.

52. The system of claim 51, wherein said unique identifier is a barcode.

53. The system of claim 52, wherein said barcode is generated using Sci-Print MP2+.

54. The system of claim 52, wherein said container is labeled with said barcode using Sci-Print MP2+.

55. The system of claim 52, wherein said automated operation comprises scanning said barcode at each step of subjecting said at least one sample to said assay protocol to generate location data for said at least one sample.

56. The system of claim 29, wherein said assay is a cell-based assay.
